(19) United States
(12) Patent Application Publication
(10) Pub. No.: US 2015/0175608 A1
(43) Pub. Date: Jun. 25, 2015

(54) NOVEL 4-SUBSTITUTED 1,3-DIHYDRO-2H-BENZIMIDAZOL-2-ONE DERIVATIVES SUBSTITUTED WITH BENZIMIDAZOLES AS RESPIRATORY SYNCYTIAL VIRUS ANTIVIRAL AGENTS

(71) Applicant: Janssen R&D Ireland, Co Cork (IE)

(72) Inventors: Abdellah Tahri, Anderlecht (BE); Tim Hugo Maria Jonckers, Heist-op-den-Berg (BE); Pierre Jean-Marie Bernard Raboisson, Rosieres (BE); Sandrine Marie Helen Vendeville, Woluwe-Saint-Pierre (BE); Lili Hu, Mechelen (BE)

(21) Appl. No.: 14/407,109

(22) PCT Filed: Jun. 14, 2013
(86) PCT No.: PCT/EP2013/062321

§ 371 Date: Dec. 11, 2014

(30) Foreign Application Priority Data
Jun. 15, 2012 (EP) 12172270.6

(51) Int. Cl. C07D 487/04; C07D 235/26

(52) U.S. Cl. CPC C07D 487/04; C07D 235/26

(57) ABSTRACT

The present invention is concerned with novel 4-substituted 1,3-dihydro-2H-benzimidazol-2-one derivatives substituted with benzimidazoles having formula (I)

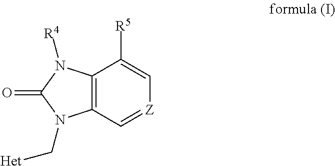

tautomers and stereoisomeric forms thereof, and the pharmaceutically acceptable addition salts, and the solvates thereof,
wherein R⁴, R⁵, Z and Het have the meaning defined in the claims. The compounds according to the present invention are useful as inhibitors on the replication of the respiratory syncytial virus (RSV). The invention further concerns the preparation of such novel compounds, compositions comprising these compounds, and the compounds for use in the treatment of respiratory syncytial virus infection.

### FIELD OF THE INVENTION

The invention concerns novel 4-substituted 1,3-dihydro-2H-benzimidazol-2-one derivatives substituted with benzimidazoles having antiviral activity, in particular, having an inhibitory activity on the replication of the respiratory syncytial virus (RSV). The invention further concerns the preparation of such novel compounds, compositions comprising these compounds, and the compounds for use in the treatment of respiratory syncytial virus infection.

### BACKGROUND

Human RSV or Respiratory Syncytial Virus is a large RNA virus, member of the family of Paramyxoviridae, subfamily pneumoviridae together with bovine RSV virus. Human RSV is responsible for a spectrum of respiratory tract diseases in people of all ages throughout the world. It is the major cause of lower respiratory tract illness during infancy and childhood. Over half of all infants encounter RSV in their first year of life, and almost all within their first two years. The infection in young children can cause lung damage that persists for years and may contribute to chronic lung disease in later life (chronic wheezing, asthma). Older children and adults often suffer from a (bad) common cold upon RSV infection. In old age, susceptibility again increases, and RSV has been implicated in a number of outbreaks of pneumonia in the aged resulting in significant mortality.

Infection with a virus from a given subgroup does not protect against a subsequent infection with an RSV isolate from the same subgroup in the following winter season. Re-infection with RSV is thus common, despite the existence of only two subtypes, A and B.

Today only three drugs have been approved for use against RSV infection. A first one is ribavirin, a nucleoside analogue that provides an aerosol treatment for serious RSV infection in hospitalized children. The aerosol route of administration, the toxicity (risk of teratogenicity), the cost and the highly variable efficacy limit its use. The other two drugs, RespiGam® (RSV-IG) and Synagis® (palivizumab), polyclonal and monoclonal antibody immunostimulants, are intended to be used in a preventive way. Both are very expensive, and require parenteral administration.

Other attempts to develop a safe and effective RSV vaccine have all met with failure thus far. Inactivated vaccines failed to protect against disease, and in fact in some cases enhanced disease during subsequent infection. Life attenuated vaccines have been tried with limited success. Clearly there is a need for an efficacious non-toxic and easy to administer drug against RSV replication. It would be particularly preferred to provide drugs against RSV replication that could be administered perorally.

A reference on benzimidazole antiviral agents is WO 01/95910. Herein compounds are presented to have antiviral activity, yet with EC₅₀ values over a wide range of from 0.001 μm to as high as 50 μM (which does not normally represent the desired biological activity). Another reference, relating to substituted 2-methyl-benzimidazole RSV antiviral agents, in the same range of activities is WO 03/053344. Another related background reference on compounds in the same range of activities, is WO 02/26228 regarding benzimidazolone antiviral agents. A reference on structure-activity relations, in respect of RSV inhibition, of 5-substituted benzimidazole compounds is X. A. Wang et al., Bioorganic and Medicinal Chemistry Letters 17 (2007) 4592-4598.

WO-2012/080446, WO-2012/080447, WO-2012/080449, WO-2012/080450 and WO-2012/080481 all filed on 16 Dec. 2011 and published on 21 Jun. 2012 disclose benzimidazole derivatives having antiviral activity against respiratory syncytial virus.

It is desired to provide new drugs that have antiviral activity. Particularly, it would be desired to provide new drugs that have RSV replication inhibitory activity. Further, it would be desired to retrieve compound structures that allow obtaining antiviral biological activities of the order of magnitude in the stronger regions of the prior art (i.e. at the bottom of the above-mentioned range of up to 50 μM), and preferably at a level of about the most active, more preferably of even stronger activity, than the compounds disclosed in the art. A further desire is to find compounds having oral antiviral activity.

### SUMMARY OF THE INVENTION

In order to better address one or more of the foregoing desires, the invention, in one aspect, presents antiviral compounds represented by formula (I),

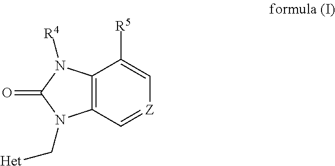

tautomers and stereoisomeric forms thereof, wherein
Het is a heterocycle having formula (a)

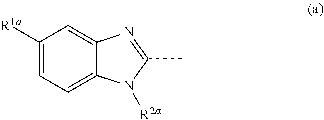

R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
R¹¹ is selected from the group consisting of C₁-C₆ alkyl, C₃-C₇cycloalkyl, phenyl, pyridinyl and pyrazolyl; each substituted with one or more substituents each independently selected from the group consisting of CF₃, CH₃, OCH₃, OCF₃ and halogen;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In another aspect, the invention relates to the foregoing compounds for use in the treatment of RSV infections in warm-blooded animals, preferably humans. In yet another aspect, the invention presents a method of treatment of viral RSV infections in a subject in need thereof, comprising administering to said subject an effective amount of a compound as defined above. In still another aspect, the invention resides in the use of a compound as defined above, for the manufacture of a medicament in the treatment of RSV infections.

In a further aspect, the invention relates to a pharmaceutical composition comprising a compound as defined above, and a pharmaceutically acceptable excipient.

In a still further aspect, the invention provides methods for preparing the compounds defined above.

### DETAILED DESCRIPTION OF THE INVENTION

The invention, in a broad sense, is based on the judicious recognition that the compounds of Formula (I) generally possess an interesting RSV inhibitory activity. Moreover, these compounds enable access to anti-RSV activities at the higher regions (lower end of the EC₅₀ values) of the range available in the aforementioned references. Particularly, on the basis of these compounds, molecular structures can be uncovered that even outperform the reference compounds in terms of biological activities.

The present invention will further be described with respect to particular embodiments and with reference to certain examples but the invention is not limited thereto but only by the claims. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Whenever the term “substituted” is used in the present invention, it is meant, unless otherwise is indicated or is clear from the context, to indicate that one or more hydrogens, in particular from 1 to 4 hydrogens, preferably from 1 to 3 hydrogens, more preferably 1 hydrogen, on the atom or radical indicated in the expression using “substituted” are replaced with a selection from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

As used herein “C₁-C₄alkyl” or “C₁₋₄alkyl” as a group or part of a group defines straight or branched chain saturated hydrocarbon radicals having from 1 to 4 carbon atoms such as methyl, ethyl, propyl, 1-methylethyl, butyl and the like.

As used herein “C₁-C₆alkyl” as a group or part of a group defines straight or branched chain saturated hydrocarbon radicals having from 1 to 6 carbon atoms such as methyl, ethyl, propyl, 1-methylethyl, butyl, pentyl, hexyl, 2-methylbutyl and the like.

“C₁-C₁₀alkyl” as a group or part of a group defines straight or branched chain saturated hydrocarbon radicals having from 1 to 10 carbon atoms such as the groups defined for C₁-C₆alkyl and heptyl, octyl, nonyl, 2-methylhexyl, 2-methylheptyl, decyl, 2-methylnonyl, and the like.

The term “C₂-C₁₀alkenyl” used herein as a group or part of a group is meant to comprise straight or branched chain unsaturated hydrocarbon radicals having at least one double bond, and preferably having one double bond, and from 2 to 10 carbon atoms such as ethenyl, propenyl, buten-1-yl, buten-2-yl, penten-1-yl, penten-2-yl, hexen-1-yl, hexen-2-yl, hexen-3-yl, 2-methylbuten-1-yl, hepten-1-yl, hepten-2-yl, hepten-3-yl, hepten-4-yl, 2-methylhexen-1-yl, octen-1-yl, octen-2-yl, octen-3-yl, octen-4-yl, 2-methylhepten-1-yl, nonen-1-yl, nonen-2-yl, nonen-3-yl, nonen-4-yl, nonen-5-yl, 2-methylocten-1-yl, decen-1-yl, decen-2-yl, decen-3-yl, decen-4-yl, decen-5-yl, 2-methylnonen-1-yl, and the like.

Whenever a “C₂-C₁₀alkenyl” group is linked to a heteroatom it preferably is linked via a saturated carbon atom.

“C₁-C₄alkyloxy” or “C₁-C₄alkoxy”, as a group or part of a group defines an O—C₁-C₄alkyl radical, wherein C₁-C₄alkyl has, independently, the meaning given above.

“C₁-C₆alkyloxy” or “C₁-C₆alkoxy”, as a group or part of a group defines an O—C₁-C₆alkyl radical, wherein C₁-C₆alkyl has, independently, the meaning given above.

The term “C₃-C₇cycloalkyl” alone or in combination, refers to a cyclic saturated hydrocarbon radical having from 3 to 7 carbon atoms. Non-limiting examples of suitable C₃-C₇cycloalkyl include cyclopropyl, cyclobutyl, cyclopentyl, cyclohexyl and cycloheptyl.

The term “—(CR^{8a}R^{9a})ₙ-” used herein defines n repetitions of the CR^{8a}R^{9a} subgroup, wherein each of these subgroups is independently defined.

The term “halo” or “halogen” as a group or part of a group is generic for fluoro, chloro, bromo, iodo unless otherwise is indicated or is clear from the context.

A term of the form NRCOOR is identical to N(R)COOR.

Examples of (but not limited to) a 4 to 6 membered aliphatic ring optionally containing one or more heteroatoms selected from the group consisting of N, S and O, as used in the definitions of R^{8a}, R^{9a} and R^{10a}, are cyclobutyl, cyclopentyl, cyclohexyl, piperidinyl, oxetanyl, tetrahydrofuranyl, tetrahydropyranyl, piperidinyl, azetidinyl, thiolanyl, piperazinyl, pyrrolidinyl.

Examples of (but not limited to) a 5 to 6 membered aromatic ring; optionally containing one or more heteroatoms selected from the group consisting of N, S and O, as used in the definition of R^{10a}, are furanyl, thienyl, pyrrolyl, oxazolyl, thiazolyl, isothiazolyl, isoxazolyl, pyrazolyl, imidazolyl, phenyl, pyridinyl, pyrimidinyl, pyrazinyl.

It should be noted that the radical positions on any molecular moiety used in the definitions may be anywhere on such moiety as long as it is chemically stable.

Radicals used in the definitions of the variables include all possible isomers unless otherwise indicated. For instance pentyl includes 1-pentyl, 2-pentyl and 3-pentyl.

When any variable occurs more than one time in any constituent, each definition is independent.

Hereinbefore and hereinafter, the term “compound(s) of formula (I)” is meant to include the tautomers and stereoisomeric forms thereof, and the pharmaceutically acceptable addition salts, and the solvates thereof.

The terms “stereoisomers”, “stereoisomeric forms” or “stereochemically isomeric forms” hereinbefore or hereinafter are used interchangeably.

The term “stereochemically isomeric forms” as used hereinbefore defines all the possible compounds made up of the same atoms bonded by the same sequence of bonds but having different three-dimensional structures which are not interchangeable, which the compounds of formula (I) may possess.

It will be appreciated that some of the compounds of formula (I) may contain one or more centers of chirality and exist as stereochemically isomeric forms.

The invention includes all stereoisomers of the compound of Formula (I) and tautomers thereof, either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. Substituents on bivalent cyclic (partially) saturated radicals may have either the cis- or trans-configuration; for example if a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof, whenever chemically possible.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

Some of the compounds according to formula (I) may also exist in their tautomeric form. Such forms although not explicitly indicated in the above formula are intended to be included within the scope of the present invention.

Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of the present invention both in pure form or in admixture with each other are intended to be embraced within the scope of the present invention.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyl-tartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of formula (I) can be obtained separately by conventional methods. Appropriate physical separation methods that may advantageously be employed are, for example, selective crystallization and chromatography, e.g. column chromatography.

For some of the compounds of formula (I), tautomers and stereoisomeric forms thereof, and the pharmaceutically acceptable addition salts, and the solvates thereof; and the intermediates used in the preparation thereof, the absolute stereochemical configuration was not experimentally determined. A person skilled in the art is able to determine the absolute configuration of such compounds using art-known methods such as, for example, X-ray diffraction.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable acid and base addition salts as mentioned hereinabove are meant to comprise the therapeutically active non-toxic acid and base addition salt forms which the compounds of formula (I) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butane-dioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term solvate comprises the hydrates and solvent addition forms which the compounds of Formula (I) are able to form, as well as the salts thereof. Examples of such forms are e.g. hydrates, alcoholates and the like.

It will be appreciated that the compounds of the invention, with reference to the aforementioned left- and right-hand parts of formula I, present a wide variety of modification.

Without detracting from the overall scope of the invention, certain embodiments are discussed in more detail below.

A compound according to the invention therefore inherently comprises a compound with one or more isotopes of one or more element, and mixtures thereof, including a radioactive compound, also called radiolabelled compound, wherein one or more non-radioactive atoms has been replaced by one of its radioactive isotopes. By the term “radiolabelled compound” is meant any compound according to Formula (I) which contains at least one radioactive atom. For example, a compound can be labelled with positron or with gamma emitting radioactive isotopes. For radioligand-binding techniques, the ³H-atom or the ¹²⁵I-atom is the atom of choice to be replaced. For imaging, the most commonly used positron emitting (PET) radioactive isotopes are ¹¹C, ¹⁸F, ¹⁵O and ¹³N, all of which are accelerator produced and have half-lives of 20, 100, 2 and 10 minutes (min) respectively. Since the half-lives of these radioactive isotopes are so short, it is only feasible to use them at institutions which have an accelerator on site for their production, thus limiting their use. The most widely used of these are ¹⁸F, ^{99m}Tc, ²⁰¹Tl and ¹²³I. The handling of these radioactive isotopes, their production, isolation and incorporation in a molecule are known to the skilled person.

In particular, the radioactive atom is selected from the group of hydrogen, carbon, nitrogen, sulfur, oxygen and halogen. In particular, the radioactive isotope is selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br.

The terms described above and others used in the specification are well understood to those in the art.

Preferred features of the compounds of this invention are now set forth.

In an embodiment, the present invention concerns novel compounds of Formula (I), tautomers and stereoisomeric forms thereof, wherein Het is a heterocycle having formula (a)

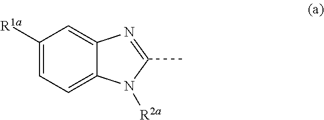

R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
R¹¹ is selected from the group consisting of C₁-C₆ alkyl, C₃-C₇cycloalkyl, phenyl, pyridinyl and pyrazolyl; each substituted with one or more substituents each independently selected from the group consisting of CF₃, CH₃, OCH₃, OCF₃ and halogen;
n is an integer having a value from 1 to 6;
R⁵ is selected from the group consisting of C₁-C₆alkyl, C₁-C₆alkyloxy, CN, CF₃ and halo;
R⁴ is selected from the group consisting of tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen; or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, N(R^{8a})CON(R^{8a}R^{9a}) and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, N(R^{8a})CON(R^{8a}R^{9a}) and C₁-C₄alkyl;
Z is CH or N;
   and the pharmaceutically acceptable addition salts, and the solvates thereof

In an embodiment, the present invention concerns novel compounds of Formula (I), tautomers and stereoisomeric forms thereof, wherein
Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
R¹¹ is selected from the group consisting of C₁-C₆ alkyl, C₃-C₇cycloalkyl, phenyl, pyridinyl and pyrazolyl; each substituted with one or more substituents each independently selected from the group consisting of CF₃, CH₃, OCH₃, OCF₃ and halogen;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂CH₃, C₁-C₄alkylcarbonyl, C₁-C₄alkyloxycarbonyl, pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), C₁-C₄alkyl, and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl;
R^{10a} is selected from the group consisting of C₁-C₆alkyl, OH, CF₃, CHF₂, F, Cl, SO₂CH₃, SO₂C₃-C₇cycloalkyl, CN, COOH;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂CH₃, C₁-C₄alkylcarbonyl, C₁-C₄alkyloxycarbonyl, pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), C₁-C₄alkyl, and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
R¹¹ is selected from the group consisting of C₁-C₆ alkyl, C₃-C₇cycloalkyl, phenyl, pyridinyl and pyrazolyl; each substituted with one or more substituents each independently selected from the group consisting of CF₃, CH₃, OCH₃, OCF₃ and halogen;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a 4 to 6 membered non-aromatic heterocycle containing one N atom, optionally being substituted with one or more substituents each independently selected from the group consisting of halo, C₁-C₄alkyloxy, SO₂R^{8a}, C₁-C₄alkylcarbonyl, CO(aryl), COHet², C₁-C₄alkyloxycarbonyl, pyridinyl, CF₃, SO₂N(C₁-C₄alkyl)₂, SO₂NH(C₁-C₄alkyl), (C═O)NH(C₁₋₄alkyl), (C═S)NH(C₁₋₄alkyl), C₁-C₄alkyl and C₁-C₄alkyl substituted with one hydroxy; or
   Het¹ represents a 4 to 6 membered non-aromatic heterocycle containing one O atom, substituted with one or more substituents each independently selected from the group consisting of halo, C₁-C₄alkyloxy, CF₃, NH(C═O)(C₁₋₄alkyl), (C═O)NH(C₁₋₄alkyl) and C₁-C₄alkyl; or
   Het¹ represents 2-azaspiro[3.3]heptyl optionally being substituted with one or more substituents each independently selected from the group consisting of halo, C₁-C₄alkyloxy, SO₂R^{8a}, C₁-C₄alkylcarbonyl, CO(aryl), COHet², C₁-C₄alkyloxycarbonyl, pyridinyl, CF₃, SO₂N(C₁-C₄alkyl)₂, SO₂NH(C₁-C₄alkyl), (C═O)NH(C₁₋₄alkyl), (C═S)NH(C₁₋₄alkyl), C₁-C₄alkyl and C₁-C₄alkyl substituted with one hydroxy;
   Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR⁸¹COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof

In an embodiment, the present invention concerns novel compounds of Formula (I), tautomers and stereoisomeric forms thereof, wherein Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of tert-butyl, C₃-C₇cycloalkyl, CH(CH₃)(CF₃), C₂-C₁₀alkenyl, CH₂CF₃, SO₂CH₃, —CH₂-p-fluorophenyl, and C₃-C₇cycloalkyl substituted with one or more substituents selected from the group consisting of halo and C₁-C₄alkyl;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a)
R^{1a} is Br or Cl;
R^{1a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is aryl or Het²; in particular aryl; also in particular Het²;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen; or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of C₃-C₇cycloalkyl, Het¹ and C₃-C₇cycloalkyl substituted with one or more substituents selected from the group consisting of halo and C₁-C₄alkyl; in particular R⁴ is Het¹;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen; or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a);
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of C₃-C₇cycloalkyl, Het¹ and C₃-C₇cycloalkyl substituted with one or more substituents selected from the group consisting of halo and C₁-C₄alkyl; in particular R⁴ is Het¹;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a)
R^{1a} is Br or Cl;
R^{1a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of CH₂CF₃, CH(CH₃)(CF₃), C₃-C₇cycloalkyl, aryl, Het¹, Het² and C₃-C₇cycloalkyl substituted with one or more substituents selected from the group consisting of halo and C₁-C₄alkyl;
   in particular C₃-C₇cycloalkyl, aryl, Het¹, Het² and C₃-C₇cycloalkyl substituted with one or more substituents selected from the group consisting of halo and C₁-C₄alkyl; more in particular aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR⁸¹COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein Het is a heterocycle having formula (a)
R^{1a} is Br or Cl;
R^{2a} is —(CR^{8a}R^{9a})ₙ—R^{10a};
each R^{8a} and R^{9a} are H;
R^{10a} is selected from the group consisting of H, OH, CF₃, CHF₂, F, Cl, SO₂CH₃, SO₂C₃-C₇cycloalkyl, NR^{8a}SO₂CH₃, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂CH₃, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂CH₃, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂CH₃, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
   or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
   and the pharmaceutically acceptable addition salts, and the solvates thereof.

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl;
R^{10a} is selected from the group consisting of H, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, and CONR^{8a}SO₂NR^{8a}R^{9a}; in particular H, OH, CF₃, CHF₂, F, Cl, SO₂CH₃, SO₂C₃-C₇cycloalkyl; more in particular SO₂CH₃, SO₂C₃-C₇cycloalkyl; even more in particular SO₂CH₃.
n is an integer having a value from 2 to 4;
R⁵ is selected from the group consisting of C₁-C₆alkyl, CF₃ and halo;
R⁴ is selected from the group consisting of C₃-C₇cycloalkyl, C₂-C₁₀alkenyl, CH(CH₃)(CF₃), CH₂CF₃ and C₃-C₇cycloalkyl substituted with one or more substituents selected from the group consisting of halo and C₁-C₄alkyl;
Z is CH or N;
   and the pharmaceutically acceptable addition salts, and the solvates thereof.+

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of hydrogen and C₁-C₁₀alkyl;
R^{10a} is selected from the group consisting of SO₂CH₃, SO₂C₃-C₇cycloalkyl, SO₂NR^{8a}R^{9a};
n is an integer having a value from 1-6; in particular 1-4; more in particular 2-4; even more in particular 3;
R⁵ is selected from the group consisting of C₁-C₆alkyl, CF₃ and halo;
R⁴ is selected from the group consisting of C₃-C₇cycloalkyl, CH₂CF₃ and C₃-C₇cycloalkyl substituted with one or more substituents selected from the group consisting of halo and C₁-C₄alkyl;
Z is CH or N;
   and the pharmaceutically acceptable addition salts, and the solvates thereof

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
R^{8a} and R^{9a} are hydrogen;
R^{10a} is selected from the group consisting of SO₂CH₃, SO₂C₃-C₇cycloalkyl, SO₂NR^{8a}R^{9a}; in particular SO₂CH₃;
n is an integer having a value from 1-6; in particular 1-4; more in particular 2-4; even more in particular 3;
R⁵ is selected from the group consisting of C₁-C₆alkyl, CF₃ and halo;
R⁴ is selected from the group consisting of C₃-C₇cycloalkyl and CH₂CF₃;
Z is CH or N;
   and the pharmaceutically acceptable addition salts, and the solvates thereof

In an embodiment, the present invention concerns novel compounds of Formula (I), tautomers and stereoisomeric forms thereof, wherein

Het is a heterocycle having formula (a)

### R^{1a} is Cl;

R^{2a} is —(CR^{8a}R^{9a})ₙ—R^{10a};
R^{8a} and R^{9a} are hydrogen;
R^{10a} is selected from the group consisting of SO₂CH₃;
n is 3;
R⁵ is selected from the group consisting of methyl, CF₃ and chloro;
R⁴ is selected from the group consisting of cyclopropyl and CH₂CF₃;

### Z is CH or N;

and the pharmaceutically acceptable addition salts, and the solvates thereof.

In an embodiment, the present invention relates to those compounds of formula (I), or any subgroup thereof as mentioned in any of the other embodiments, wherein
R⁵ is selected from the group consisting of C₁-C₆alkyl, CF₃ and halo;
R⁴ is selected from the group consisting of C₃-C₇cycloalkyl and CH₂CF₃; in particular C₃-C₇cycloalkyl; more in particular cyclopropyl;
Z is CH or N.

In an embodiment, the present invention concerns novel compounds of Formula (I), tautomers and stereoisomeric forms thereof, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of H and C₁-C₁₀alkyl; or R^{8a} and R^{9a} taken together form a 4 to 6 membered aliphatic ring that optionally contains a heteroatom selected from the group N, S and O;
R^{10a} is selected from the group consisting of H, OH, CF₃, CHF₂, F, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a} and OCOC₁-C₆alkyl;
n is an integer having a value from 2 to 6;
R⁵ is selected from the group consisting of C₁-C₆alkyl, C₁-C₆alkoxy, halogen and CN;
R⁴ is selected from the group consisting of H, C₃-C₇cycloalkyl, C₂-C₁₀alkenyl, and SO₂CH₃; in particular R⁴ is selected from the group consisting of C₃-C₇cycloalkyl, C₂-C₁₀alkenyl and SO₂CH₃;
aryl represents phenyl or naphthalenyl; said aryl optionally being substituted with one or more substituents each independently selected from the group consisting of halo, C₁-C₄alkyloxy, SO₂R^{8a}, SO₂NR^{8a}R^{9a}, CF₃ and C₁-C₄alkyl; in particular the substituents each independently are selected from the group consisting of halo, C₁-C₄alkyloxy, SO₂CH₃, SO₂N(C₁-C₄alkyl)₂, SO₂NH(C₁-C₄alkyl), CF₃ and C₁-C₄alkyl;
Het¹ represents a monocyclic 5 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CF₃, SO₂N(C₁-C₄alkyl)₂, SO₂NH(C₁-C₄alkyl), (C═O)NH(C₁₋₄alkyl) and C₁-C₄alkyl;
Het² represents a monocyclic 5 to 6 membered aromatic heterocycle containing one or two heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, SO₂R^{8a}, CF₃, SO₂N(C₁-C₄alkyl)₂, SO₂NH(C₁-C₄alkyl), (C═O)NH(C₁₋₄alkyl) and C₁-C₄alkyl;
Z is CH or N;
   and the pharmaceutically acceptable addition salts, and the solvates thereof.

In an embodiment, the present invention relates to those compounds of formula (I), or any subgroup thereof as mentioned in any of the other embodiments, wherein R⁴ is other than hydrogen.

In an embodiment, the present invention relates to those compounds of formula (I), or any subgroup thereof as mentioned in any of the other embodiments, wherein
R⁵ is selected from the group consisting of C₁-C₆alkyl, CF₃ and halo;
R⁴ is selected from the group consisting of C₃-C₇cycloalkyl, CH₂CF₃ and C₃-C₇cycloalkyl substituted with one or more substituents selected from the group consisting of halo and C₁-C₄alkyl; in particular R⁴ is selected from the group consisting of C₃-C₇cycloalkyl and CH₂CF₃;
Z is CH or N.

In an embodiment, the present invention relates to those compounds of formula (I), or any subgroup thereof as mentioned in any of the other embodiments, wherein
aryl represents phenyl or naphthalenyl; said aryl optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂CH₃, C₁-C₄alkylcarbonyl, C₁-C₄alkyloxycarbonyl, pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), C₁-C₄alkyl, and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, SO₂CH₃, CF₃, SO₂N(C₁-C₄alkyl)₂, SO₂NH(C₁-C₄alkyl), CN, (C═O)NH(C₁-C₄alkyl), (C═O)N(C₁-C₄alkyl)₂, NH(C═O)O(C₁₋₄alkyl), O(C═O)NH(C₁-C₄alkyl), O(C═O)N(C₁-C₄alkyl)₂ and C₁-C₄alkyl.

In an embodiment, the present invention relates to those compounds of formula (I), or any subgroup thereof as mentioned in any of the other embodiments, wherein
Het¹ represents a 4 to 6 membered non-aromatic heterocycle containing one N atom, optionally being substituted with one or more substituents each independently selected from the group consisting of halo, C₁-C₄alkyloxy, SO₂R^{8a}, C₁-C₄alkylcarbonyl, CO(aryl), COHet², C₁-C₄alkyloxycarbonyl, pyridinyl, CF₃, SO₂N(C₁-C₄alkyl)₂, SO₂NH(C₁-C₄alkyl), (C═O)NH(C₁₋₄alkyl), (C═S)NH(C₁₋₄alkyl), C₁-C₄alkyl and C₁-C₄alkyl substituted with one hydroxy; or
   Het¹ represents a 4 to 6 membered non-aromatic heterocycle containing one O atom, substituted with one or more substituents each independently selected from the group consisting of halo, C₁-C₄alkyloxy, CF₃, NH(C═O)(C₁₋₄alkyl), (C═O)NH(C₁₋₄alkyl) and C₁-C₄alkyl; or
   Het¹ represents 2-azaspiro[3.3]heptyl optionally being substituted with one or more substituents each independently selected from the group consisting of halo, C₁-C₄alkyloxy, SO₂R^{8a}, C₁-C₄alkylcarbonyl, CO(aryl), COHet², C₁-C₄alkyloxycarbonyl, pyridinyl, CF₃, SO₂N(C₁-C₄alkyl)₂, SO₂NH(C₁-C₄alkyl), (C═O)NH(C₁₋₄alkyl), (C═S)NH(C₁₋₄alkyl), C₁-C₄alkyl and C₁-C₄alkyl substituted with one hydroxy.

In an embodiment, the present invention relates to those compounds of formula (I), or any subgroup thereof as mentioned in any of the other embodiments, wherein each R^{8a} and R^{9a} are independently chosen from the group consisting of H, C₁-C₁₀alkyl and C₃-C₇cycloalkyl; in particular C₁-C₁₀alkyl and C₃-C₇cycloalkyl.

In an embodiment, the present invention relates to those compounds of formula (I), or any subgroup thereof as mentioned in any of the other embodiments, wherein each R^{8a} and R^{9a} are independently chosen from the group consisting of H, C₁-C₁₀alkyl and C₃-C₇cycloalkyl;
R^{10a} is selected from the group consisting of C₁-C₆alkyl, OH, CF₃, CHF₂, F, Cl, SO₂CH₃, SO₂C₃-C₇cycloalkyl, CN, COOH; in particular SO₂CH₃.

In an embodiment, the present invention relates to those compounds of formula (I), or any subgroup thereof as mentioned in any of the other embodiments, wherein R^{10a} is selected from the group consisting of H, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O; in particular wherein R^{10a} is selected from the group consisting of H, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O.

In an embodiment, the present invention relates to those compounds of formula (I), or any subgroup thereof as mentioned in any of the other embodiments, wherein Het¹ represents a monocyclic 4 to 6 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl),

(C═O)NH(C₁₋₄alkyl), (C═S)NH(C₁₋₄alkyl), C₁-C₄alkyl and C₁-C₄alkyl substituted with one hydroxy.

In an embodiment, the present invention relates to those compounds of formula (I), or any subgroup thereof as mentioned in any of the other embodiments, wherein R⁴ is selected from the group consisting of tert-butyl, C₃-C₇cycloalkyl, CH(CH₃)(CF₃), C₂-C₁₀alkenyl, CH₂CF₃, SO₂CH₃, —CH₂-p-fluorophenyl, and C₃-C₇cycloalkyl substituted with one or more substituents selected from the group consisting of halo and C₁-C₄alkyl.

In an embodiment, the present invention relates to those compounds of formula (I), or any subgroup thereof as mentioned in any of the other embodiments, wherein R⁴ is aryl or Het².

In an embodiment, the present invention relates to those compounds of formula (I), or any subgroup thereof as mentioned in any of the other embodiments, wherein R⁴ is cyclopropyl or CH₂CF₃; in particular CH₂CF₃.

In an embodiment, the present invention relates to those compounds of formula (I), or any subgroup thereof as mentioned in any of the other embodiments, wherein R⁴ is selected from the group consisting of tert-butyl, CH(CH₃)(CF₃), CH₂CF₃, aryl, Het¹, Het² and C₃-C₇cycloalkyl substituted with one or more substituents selected from the group consisting of halo and C₁-C₄alkyl; in particular wherein R⁴ is selected from the group consisting of tert-butyl, CH(CH₃)(CF₃), aryl, Het¹, Het² and C₃-C₇cycloalkyl substituted with one or more substituents selected from the group consisting of halo and C₁-C₄alkyl.

In an embodiment, the present invention relates to those compounds of formula (I), or any subgroup thereof as mentioned in any of the other embodiments, wherein R⁴ is aryl.

In an embodiment, the present invention relates to those compounds of formula (I), or any subgroup thereof as mentioned in any of the other embodiments, wherein R⁴ is Het².

In an embodiment, the present invention relates to those compounds of formula (I), or any subgroup thereof as mentioned in any of the other embodiments, wherein R⁴ is selected from the group consisting of C₃-C₇cycloalkyl, Het¹ and C₃-C₇cycloalkyl substituted with one or more substituents selected from the group consisting of halo and C₁-C₄alkyl.

In an embodiment, the present invention relates to those compounds of formula (I), or any subgroup thereof as mentioned in any of the other embodiments, wherein R⁴ is selected from the group consisting of C₃-C₇cycloalkyl and C₃-C₇cycloalkyl substituted with one or more substituents selected from the group consisting of halo and C₁-C₄alkyl.

In an embodiment, the present invention relates to those compounds of formula (I), or any subgroup thereof as mentioned in any of the other embodiments, wherein R⁴ is Het¹.

In an embodiment, the present invention relates to those compounds of formula (I), or any subgroup thereof as mentioned in any of the other embodiments, wherein R⁵ is selected from the group consisting of methyl, chloro and CF₃; and wherein R⁴ is cyclopropyl.

In an embodiment, the present invention relates to those compounds of formula (I), or any subgroup thereof as mentioned in any of the other embodiments, wherein R⁴ is cyclopropyl.

In an embodiment, the present invention relates to those compounds of formula (I), or any subgroup thereof as mentioned in any of the other embodiments, wherein R⁴ is selected from the group consisting of C₃-C₇cycloalkyl, CH₂CF₃ and C₃-C₇cycloalkyl substituted with one or more substituents selected from the group consisting of halo and C₁-C₄alkyl; in particular R⁴ is selected from the group consisting of C₃-C₇cycloalkyl and CH₂CF₃.

In an embodiment, the present invention relates to those compounds of formula (I), or any subgroup thereof as mentioned in any of the other embodiments, wherein R^{8a} and R^{9a} are hydrogen.

In an embodiment, the present invention relates to those compounds of formula (I), or any subgroup thereof as mentioned in any of the other embodiments, wherein R^{10a} is SO₂CH₃.

In an embodiment, the present invention relates to those compounds of formula (I), or any subgroup thereof as mentioned in any of the other embodiments, wherein R⁴ is selected from the group consisting of C₃-C₇cycloalkyl, aryl, Het¹, Het² and C₃-C₇cycloalkyl substituted with one or more substituents selected from the group consisting of halo and C₁-C₄alkyl.

In an embodiment, the present invention relates to those compounds of formula (I), or any subgroup thereof as mentioned in any of the other embodiments, wherein R^{1a} is Br.

In an embodiment, the present invention relates to those compounds of formula (I), or any subgroup thereof as mentioned in any of the other embodiments, wherein R^{1a} is Cl.

In an embodiment, the present invention relates to those compounds of formula (I), or any subgroup thereof as mentioned in any of the other embodiments, wherein both R^{8a} and R^{9a} are H; and wherein n is 2-4, preferably n is 3 or 4.

In an embodiment, the present invention relates to those compounds of formula (I), or any subgroup thereof as mentioned in any of the other embodiments, wherein n is 2-4.

In an embodiment, the present invention relates to those compounds of formula (I), or any subgroup thereof as mentioned in any of the other embodiments, wherein n is 3-4.

In an embodiment, the present invention relates to those compounds of formula (I), or any subgroup thereof as mentioned in any of the other embodiments, wherein R^{10a} is selected from the group consisting of H, OH, F, CF₃, CN and SO₂CH₃; in particular SO₂CH₃.

In an embodiment, the present invention relates to those compounds of formula (I), or any subgroup thereof as mentioned in any of the other embodiments, wherein R₄ is selected from the group consisting of C₃-C₇cycloalkyl, C₂-C₁₀alkenyl, CH₂CF₃ and SO₂CH₃; preferably C₃-C₇cycloalkyl, C₂-C₁₀alkenyl and SO₂CH₃; more preferably R₄ is C₃-C₇cycloalkyl or CH₂CF₃; even more preferably R₄ is cyclopropyl or CH₂CF₃.

In an embodiment, the present invention relates to those compounds of formula (I), or any subgroup thereof as mentioned in any of the other embodiments, wherein Z is N.

In another embodiment, the present invention relates to those compounds of formula (I), or any subgroup thereof as mentioned in any of the other embodiments, wherein Z is CH.

In an embodiment, the present invention relates to those compounds of formula (I), or any subgroup thereof as mentioned in any of the other embodiments, wherein R₅ is selected from the group consisting of C₁-C₆alkyl, C₁-C₆alkyloxy, halogen, and CN.

In an embodiment, the present invention relates to those compounds of formula (I), or any subgroup thereof as mentioned in any of the other embodiments, wherein R₅ is selected from the group consisting of C₁-C₆alkyl, C₁-C₆alkyloxy, halogen and CF₃; in particular C₁-C₆alkyl, halogen and CF₃; more in particular chloro, CF₃ and methyl; even more in particular chloro and methyl.

Preferred compounds are compounds P1-P7, stereoisomeric forms thereof, and pharmaceutically acceptable addition salts, free bases and solvates thereof.

### General Synthetic Schemes

The compounds of formula I may be prepared by the methods described below, using synthetic methods known in the art of organic chemistry, or modifications and derivatisations that are familiar to those skilled in the art. The starting materials used herein are commercially available or may be prepared by routine methods known in the art such as those methods disclosed in standard reference books. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction schemes discussed herein below. Unless otherwise indicated, the substituents in the schemes are defined as above. Isolation and purification of the products is accomplished by standard procedures, which are known to a chemist of ordinary skill.

Scheme 1 illustrates a method for the preparation of compounds of formula I, where R^{1a}, R^{2a}, R⁴, R⁵ and Z are defined as above.

The compounds of formula (I) can be synthesized for instance using one of the methods shown in Scheme 1. In general, a fragment A or B is coupled with a fragment C resulting in derivatives of formula (I).

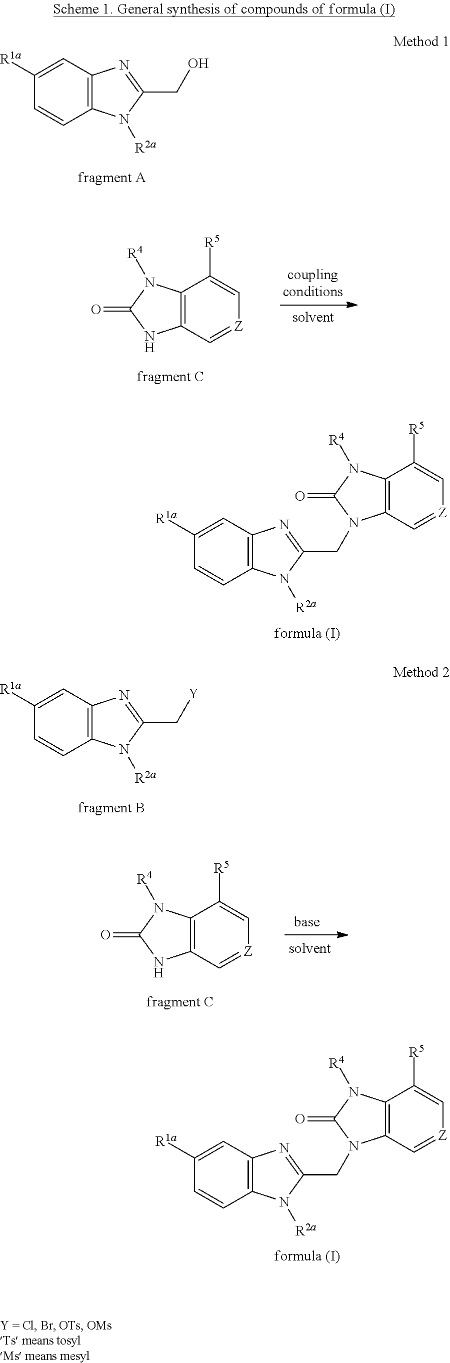

For method 1, an example of suitable “coupling conditions” to react a fragment A with a fragment C to form formula (I) type compounds is a Mitsunobu reaction. A suitable solvent for this type of reaction is for example THF (tetrahydrofuran).

Alternatively (but not limited to), a fragment B type intermediate wherein Z═Cl, Br, OTs (tosylate), OMs (mesylate) can be reacted with a fragment C type intermediate through a base mediated coupling reaction. (Method 2) Possible bases to effect this reaction (but not limited to) are K₂CO₃, Cs₂CO₃, triethylamine, sodium hydride. A suitable solvent (but not limited to) for this type of base mediated coupling is DMF (dimethylformamide).

Fragment A type intermediates can be generally prepared as depicted in scheme 2.

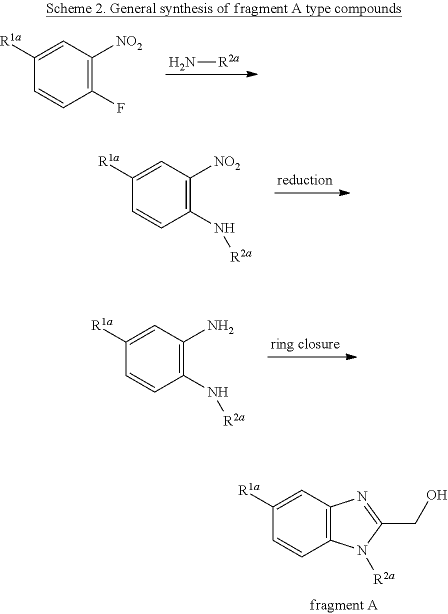

In general, fragment B type intermediates can be prepared from fragment A type intermediates through reaction with reagents like (but not limited to) SOCl₂, PBr₃, p-TsCl (4-toluenesulfonyl chloride), MsCl (methanesulfonyl chloride).

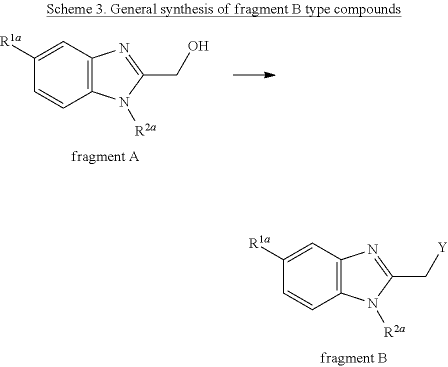

Fragment C type intermediates of formula (III) can be prepared as depicted in Scheme 4.

The Synthesis of 2-oxo-imidazopyridine derivatives and 2-oxo-imidazobenzene derivatives is shown in scheme 4. Intermediates of formula (III) can be synthesized using the procedure depicted in scheme 4. Displacement of W, which is a halide, preferably fluorine, or an alkyloxy group, preferably methoxy, of the nitro pyridine or of nitro aryl of formula (XVII) with an amine, in a suitable solvent such as THF or DMF, in the presence of an organic base such as triethyl amine or diisopropyl ethyl amine, gives an intermediate of formula (XVIII). Reduction of the nitro group to the amine (XIX) can be done in a catalytic way using hydrogen in the presence of a catalyst such as palladium or platinum, in a suitable solvent such as methanol, or in a stoichiometric way using iron in the presence of ammonium chloride or tin chloride in the presence of concentrated hydrochloric acid. The cyclisation of the resulting diamine (XIX) using CDI (1,1′-carbonyldiimidazole), phosgene or triphosgene, in a solvent such as acetonitrile or THF, provides the N³-substituted 2-oxo-imidazopyridine or N³-substituted 2-oxo-imidazobenzene of formula (III). Alternatively, the intermediate of formula (III) may be prepared starting from commercially available dianilines (XX) which can be cyclized by ring closure with CDI, phosgene or triphosgene to intermediates of type (XXI). Introduction of a R⁴ substituent (R⁴ not being H) on an intermediate of formula XXI can be accomplished by a Mitsunobu reaction with commercially available alcohols, or by displacement of the LG in the intermediates of formula XXII, where LG is a leaving group such as halide, preferably bromine, or sulfonate, in the presence of a base such as sodium hydride, potassium carbonate or cesium carbonate in a suitable solvent such as DMF or THF. This will finally yield intermediates of formula III.

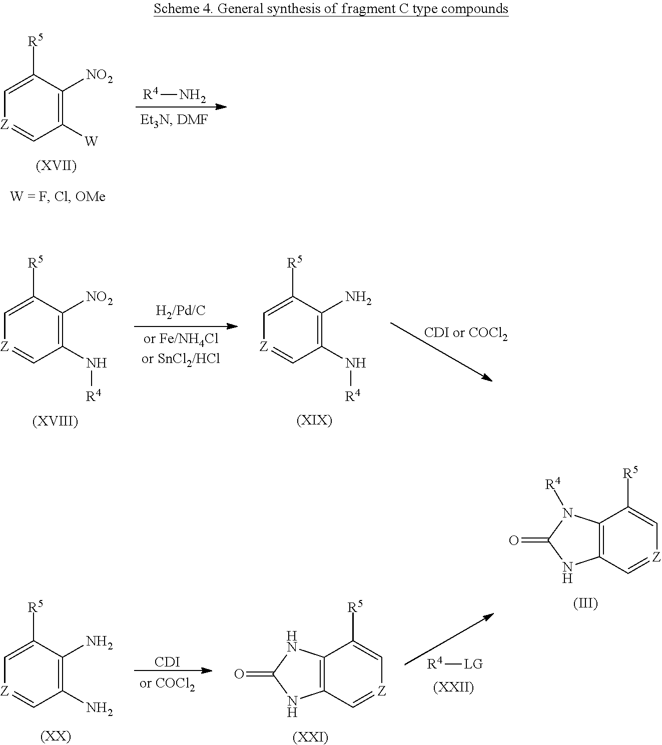

All starting materials can be obtained commercially or can be prepared by those skilled in the art.

Pure stereochemically isomeric forms of the compounds of formula (I) may be obtained by the application of art-known procedures. Diastereomers may be separated by physical methods such as selective crystallization and chromatographic techniques, e.g., counter-current distribution, liquid chromatography and the like. The compounds of formula (I) as prepared in the hereinabove described processes are generally racemic mixtures of enantiomers which can be separated from one another following art-known resolution procedures. The racemic compounds of formula (I) which are sufficiently basic or acidic may be converted into the corresponding diastereomeric salt forms by reaction with a suitable chiral acid, respectively chiral base. Said diastereomeric salt forms are subsequently separated, for example, by selective or fractional crystallization and the enantiomers are liberated therefrom by alkali or acid. An alternative manner of separating the enantiomeric forms of the compounds of formula (I) involves liquid chromatography, in particular liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

In a further aspect, the present invention concerns a pharmaceutical composition comprising a therapeutically effective amount of a compound of formula (I) as specified herein, or a compound of any of the embodiments of compounds of formula (I) as specified herein, and a pharmaceutically acceptable carrier. A therapeutically effective amount in this context is an amount sufficient to prophylaxictically act against, to stabilize or to reduce viral infection, and in particular RSV viral infection, in infected subjects or subjects being at risk of being infected. In still a further aspect, this invention relates to a process of preparing a pharmaceutical composition as specified herein, which comprises intimately mixing a pharmaceutically acceptable carrier with a therapeutically effective amount of a compound of formula (I), as specified herein, or of a compound of any of the embodiments of compounds of formula (I) as specified herein.

Therefore, the compounds of the present invention or any embodiment thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

The compounds of the present invention may also be administered via oral inhalation or insufflation by means of methods and formulations employed in the art for administration via this way. Thus, in general the compounds of the present invention may be administered to the lungs in the form of a solution, a suspension or a dry powder, a solution being preferred. Any system developed for the delivery of solutions, suspensions or dry powders via oral inhalation or insufflation are suitable for the administration of the present compounds.

Thus, the present invention also provides a pharmaceutical composition adapted for administration by inhalation or insufflation through the mouth comprising a compound of formula (I) and a pharmaceutically acceptable carrier. Preferably, the compounds of the present invention are administered via inhalation of a solution in nebulized or aerosolized doses.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The compounds of formula (I) show antiviral properties. Viral infections treatable using the compounds and methods of the present invention include those infections brought on by ortho- and paramyxoviruses and in particular by human and bovine respiratory syncytial virus (RSV). A number of the compounds of this invention moreover are active against mutated strains of RSV. Additionally, many of the compounds of this invention show a favorable pharmacokinetic profile and have attractive properties in terms of bioavailability, including an acceptable half-life, AUC and peak values and lacking unfavourable phenomena such as insufficient quick onset and tissue retention.

The in vitro antiviral activity against RSV of the present compounds was tested in a test as described in the experimental part of the description, and may also be demonstrated in a virus yield reduction assay. The in vivo antiviral activity against RSV of the present compounds may be demonstrated in a test model using cotton rats as described in Wyde et al. (Antiviral Research (1998), 38, 31-42).

Due to their antiviral properties, particularly their anti-RSV properties, the compounds of formula (I) or any embodiment thereof, tautomers and stereoisomeric forms thereof, and the pharmaceutically acceptable addition salts, and the solvates thereof, are useful in the treatment of individuals experiencing a viral infection, particularly a RSV infection, and for the prophylaxis of these infections. In general, the compounds of the present invention may be useful in the treatment of warm-blooded animals infected with viruses, in particular the respiratory syncytial virus.

The compounds of the present invention or any embodiment thereof may therefore be used as medicines. Said use as a medicine or method of treatment comprises the systemic administration to viral infected subjects or to subjects susceptible to viral infections of an amount effective to combat the conditions associated with the viral infection, in particular the RSV infection.

The present invention also relates to the use of the present compounds or any embodiment thereof in the manufacture of a medicament for the treatment or the prevention of viral infections, particularly RSV infection.

The present invention furthermore relates to a method of treating a warm-blooded animal infected by a virus, or being at risk of infection by a virus, in particular by RSV, said method comprising the administration of an anti-virally effective amount of a compound of formula (I), as specified herein, or of a compound of any of the embodiments of compounds of formula (I), as specified herein.

In general it is contemplated that an antivirally effective daily amount would be from 0.01 mg/kg to 500 mg/kg body weight, more preferably from 0.1 mg/kg to 50 mg/kg body weight. It may be appropriate to administer the required dose as two, three, four or more sub-doses at appropriate intervals throughout the day. Said sub-doses may be formulated as unit dosage forms, for example, containing 1 to 1000 mg, and in particular 5 to 200 mg of active ingredient per unit dosage form.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention. The effective daily amount ranges mentioned hereinabove are therefore only guidelines.

Also, the combination of another antiviral agent and a compound of formula (I) can be used as a medicine. Thus, the present invention also relates to a product containing (a) a compound of formula (I), and (b) another antiviral compound, as a combined preparation for simultaneous, separate or sequential use in antiviral treatment. The different drugs may be combined in a single preparation together with pharmaceutically acceptable carriers. For instance, the compounds of the present invention may be combined with interferon-beta or tumor necrosis factor-alpha in order to treat or prevent RSV infections.

The invention will hereinafter be illustrated with reference to the following, non-limiting examples.

### Experimental Part

Hereinafter, the term ‘eq.’ means equivalent, ‘THF’ means tetrahydrofuran, ‘Psi’ means pound-force per square inch, ‘DMF’ means N,N-dimethylformamide, ‘DMSO’ means dimethyl sulfoxide, ‘DIEA’ means diisopropylethylamine, ‘DIAD’ means diisopropyl azodicarboxylate, ‘HOAc’ or ‘AcOH’ means acetic acid, ‘RP’ means reversed phase, ‘EtOAc’ means ethyl acetate, ‘Pd(dppf)Cl₂CH₂Cl₂’ means [1,1′-bis-(diphenylphosphino)ferrocene]palladium chloride complex with dichloromethane, ‘TPP’ means triphenylphosphine, ‘m-cPBA’ means 3-chlorobenzenecarboperoxoic acid, ‘Cu(OAc)₂’ means copper(II) acetate, ‘EtOH’ means ethanol, ‘MeOH’ means methanol, ‘MeCN’ means methyl cyanide, ‘CDI’ means 1,1′-carbonyldiimidazole, ‘KOEt’ means potassium ethoxide, and ‘HPLC’ means High Performance Liquid Chromatography.

LCMS (Liquid Chromatography/Mass spectrometry)

LCMS was done using either of the following methods:

### General Method A

The LC measurement was performed using an Acquity UPLC (Waters) (‘UPLC’ means Ultra Performance Liquid Chromatography) system comprising a binary pump, a sample organizer, a column heater (set at 55° C.), a diode-array detector (DAD) and a column as specified in the respective methods below. Flow from the column was split to a MS spectrometer. The MS detector was configured with an electrospray ionization source. Mass spectra were acquired by scanning from 100 to 1000 in 0.18 seconds using a dwell time of 0.02 seconds. The capillary needle voltage was 3.5 kV and the source temperature was maintained at 140° C. Nitrogen was used as the nebulizer gas. Data acquisition was performed with a Waters-Micromass MassLynx-Openlynx data system.

### General Method B

The LC measurement was performed using an Acquity UPLC (Waters) system comprising a binary pump, a sample organizer, a column heater (set at 55° C.), a diode-array detector (DAD) and a column as specified in the respective methods below. All the flow from the column went to a MS spectrometer. The MS detector was configured with an electrospray ionization source. Mass spectra were acquired by scanning from 120 to 1000 in 0.1 seconds. The capillary needle voltage was 3.0 kV and the source temperature was maintained at 150° C. Nitrogen was used as the nebulizer gas. Data acquisition was performed with a Waters-Micromass MassLynx-Openlynx data system.

### Method 1

In addition to the general method A: Reversed phase UPLC was carried out on a bridged ethylsiloxane/silica hybrid (BEH) C18 column (1.7 μm, 2.1×50 mm; Waters Acquity) with a flow rate of 0.8 ml/min. Two mobile phases (10 mM ammonium acetate in H₂O/acetonitrile 95/5; mobile phase B: acetonitrile) were used to run a gradient condition from 95% A and 5% B to 5% A and 95% B in 1.3 minutes and hold for 0.3 minutes. An injection volume of 0.5 μl was used. Cone voltage was 10 V for positive ionization mode and 20 V for negative ionization mode.

### Method 2

In addition to the general method A: Reversed phase UPLC (Ultra Performance Liquid Chromatography) was carried out on a bridged ethylsiloxane/silica hybrid (BEH) C18 column (1.7 μm, 2.1×50 mm; Waters Acquity) with a flow rate of 0.8 ml/min. Two mobile phases (mobile phase A: 0.1% formic acid in H₂O/methanol 95/5; mobile phase B: methanol) were used to run a gradient condition from 95% A and 5% B to 5% A and 95% B in 1.3 minutes and hold for 0.2 minutes. An injection volume of 0.5 μl was used. Cone voltage was 10 V for positive ionization mode and 20 V for negative ionization mode.

### Method 3

In addition to the general method B: In addition to the general method B: Reversed phase UPLC (Ultra Performance Liquid Chromatography) was carried out on a Acquity UPLC HSS T3 column (1.8 μm, 2.1×100 mm; Waters Acquity) with a flow rate of 0.8 ml/min. Two mobile phases (A: 10 mM ammonium acetate in H₂O/acetonitrile 95/5; mobile phase B: acetonitrile) were used to run a gradient condition from 95% A and 5% B to 0% A and 100% B in 2.5 minutes and subsequently to 5% A and 95% B in 0.5 minutes. An injection volume of 1 μl was used. Cone voltage was 30 V for positive ionization mode and 30 V for negative ionization mode.

### NMR

For a number of compounds, ¹H NMR spectra were recorded on a Bruker DPX-400 spectrometer operating at 400 MHz or on a Bruker DPX-360 operating at 360 MHz using CHLOROFORM-d (deuterated chloroform, CDCl₃) or DMSO-d₆ (deuterated DMSO, dimethyl-d6 sulfoxide) as solvent. Chemical shifts (δ) are reported in parts per million (ppm) relative to tetramethylsilane (TMS), which was used as internal standard.

### Melting Points

For a number of compounds, melting points (m.p.) were determined with a DSC823e (Mettler-Toledo). Melting points were measured with a temperature gradient of 30° C./minute. Maximum temperature was 400° C. Values are peak values.

### Synthesis of Intermediates

All the intermediates needed for the synthesis of targeted compounds of formula (I) are synthesized as described in the following schemes 5 to 13.

The invention will hereinafter be illustrated with reference to the following, non-limiting examples.

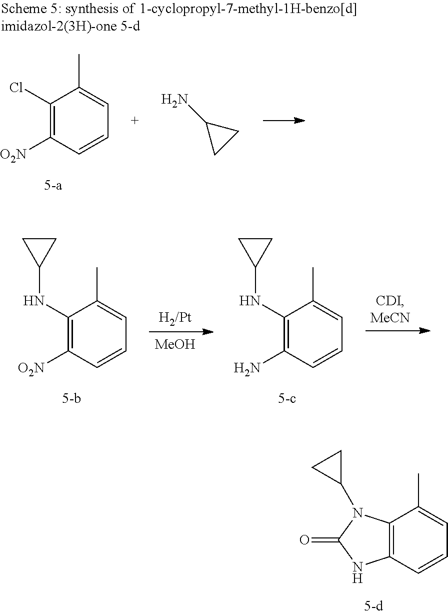

### Step 1: Synthesis of N-cyclopropyl-2-methyl-6-nitroaniline 5-b

The mixture of 2-chloro-1-methyl-3-nitrobenzene 5-a (30 g, 174.8 mmol, 5 eq.) and cyclopropylamine (50 g, 874 mmol, 5 eq.) was stirred in a sealed tube at 120° C. for 2 days. The mixture was cooled to room temperature. Then water (100 mL) was added. The aqueous layer was extracted with CH₂Cl₂ (3×100 mL). The combined organic layers were washed with brine, dried over Na₂SO₄ and concentrated. The residue was purified by preparative high-performance liquid chromatography (column C18, eluent: CH₃CN/H₂O from 55/45 to 71.4/28.6, 0.1% CF₃COOH). The desired fractions were collected and the organic solvent was removed under vacuum. The aqueous solution was neutralized to pH=7-8 with aqueous NaHCO₃ solution and extracted with ethyl acetate. The combined organic layer was washed with brine, dried over Na₂SO₄ and concentrated to give the desired product: 13 g of intermediate 5-b was obtained (37.9% yield).

### Step 2: Synthesis of N¹-cyclopropyl-6-methylbenzene-1,2-diamine 5-c

Intermediate 5-b (13 g, 67.6 mmol) in methanol (50 mL), THF (50 mL) and ethyl acetate (50 mL) was hydrogenated (50 Psi) at 25° C. with Pt/C (1.3 g) as a catalyst for 3 hours. After uptake of H₂ (3 eq.), the catalyst was filtered off and the filtrate was evaporated. The residue was purified by column chromatography over silica gel (eluent: CH₂Cl₂/CH₃OH from 100/1 to 50/1). 6.2 g of intermediate 5-c was obtained (Yield 56%).

### Step 3: Synthesis of 1-cyclopropyl-7-methyl-1H-benzo[d]imidazol-2(3H)-one 5-d

Carbonyldiimidazole (6.5 g, 40.1 mmol, 1.05 eq.) was added to a solution of intermediate 5-c (6.2 g, 38.2 mmol, 1 eq.) in CH₃CN (60 mL) at 0° C. The reaction mixture was allowed to warm to 25° C. and stirred for 1 h. The solid was collected by filtration and was washed with CH₃CN (15 mL) to afford intermediate 5-d as a white powder (2.6 g, 35%).

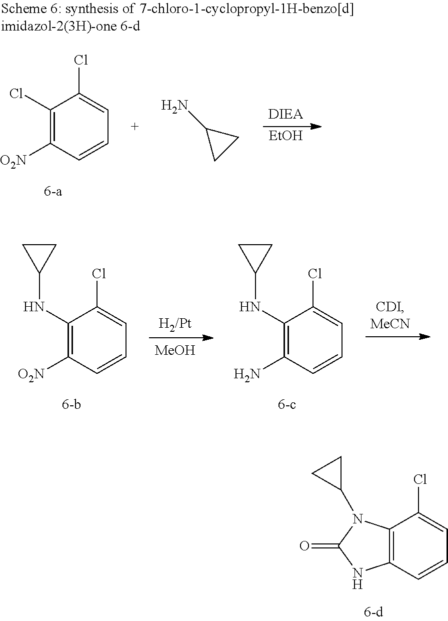

### Step 1: Synthesis of 2-chloro-N-cyclopropyl-6-nitroaniline 6-b

Cyclopropylamine (11.9 g, 208 mmol, 2 eq.) was added dropwise to a solution of 1,2 dichloro-3-nitrobenzene 6-a (20 g, 104 mmol, 1 eq.) and diisopropyl ethyl amine (26.9 g, 208 mmol, 2 eq.) in ethanol (300 mL) at 0° C. The resulting mixture was refluxed for 3 days. The mixture was cooled down to room temperature and filtered. The solid was washed with cooled ethanol and dried under vacuum. The intermediate 6-b was isolated as a solid (10 g, 45%).

### Step 2: Synthesis of 6-chloro-N¹-cyclopropylbenzene-1,2-diamine 6-c

Intermediate 6-b (10 g, 47 mmol) in methanol (35 mL), THF (35 mL) and ethyl acetate (35 mL) was hydrogenated (50 Psi) at 25° C. with wet Pt/C (1 g) as a catalyst for 12 hours. After uptake of H₂ (3 eq), the catalyst was filtered off and the filtrate was evaporated. The intermediate 6-c was obtained (8 g, Yield 56%).

### Step 3: Synthesis of 7-chloro-1-cyclopropyl-1H-benzo[d]imidazol-2(3H)-one 6-d

Carbonyldiimidazole (8 g, 42 mmol, 1.02 eq.) was added to a solution of intermediate 5-c (7.5 g, 41 mmol, 1 eq.) in CH₃CN (80 mL) at 0° C. The reaction mixture was allowed to warm to 25° C. and stirred for 1 h. The solid was collected by filtration and was washed with CH₃CN (15 mL) to afford intermediate 6-d as a white powder (2.5 g, 25%).

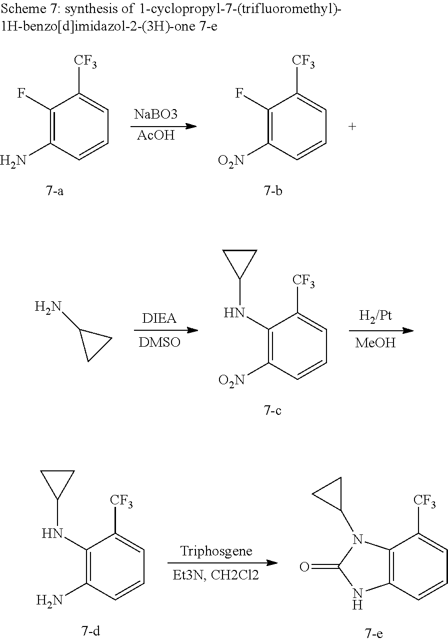

### Step 1: Synthesis of 2-fluoro-1-nitro-3-(trifluoromethyl)benzene 7-b

To a solution of 2-fluoro-3-(trifluoromethyl)aniline 7-a (11.6 g, 64.8 mmol, 1 eq.) in acetic acid (120 mL) sodium perborate monohydrate (12.3 g, 64.8 mmol, 1 eq.) in acetic acid (240 mL) was added dropwise at 55° C. over 6 h. After being stirred at 55° C. for an additional 9 h, the solution was allowed to cool to room temperature and filtered. The filtrate was concentrated to 60 ml under vacuum below 50° C. The residue was dissolved in ethyl acetate (500 mL), washed with water (150 ml×3), aqueous NaHCO₃ solution (150 mL×2) and brine, and then dried over MgSO₄, and concentrated in vacuum. 11.2 g of intermediate 7-b was obtained (yield 50%).

### Step 2: Synthesis of N-cyclopropyl-2-nitro-6-(trifluoromethyl)aniline 7-c

The mixture of intermediate 7-b (11.2 g, purity 60%, 32 mmol, 1 eq.), cyclopropylamine (3.67 g, 64 mmol, 2 eq.) and diisopropylethyl amine (8.3 g, 64 mmol, 2 eq.) in DMSO (110 mL) was stirred overnight at 70° C. Ethyl acetate (300 mL) was added to the solution and washed with water (100 mL×5). The organic layer was dried over Na₂SO₄ and concentrated. The residue was purified by preparative high-performance liquid chromatography (column C18, eluent: CH₃CN, H₂O form 25/75 to 35/65, 0.1% CF₃COOH). The desired fractions were collected, neutralized with aqueous NaHCO₃ and concentrated under vacuum. 2.4 g of intermediate 7-c was obtained (Yield 30%).

### Step 3: Synthesis of N¹-cyclopropyl-6-(trifluoromethyl)benzene-1,2-diamine 7-d

Intermediate 7-c (2.4 g, 9.7 mmol) in methanol (100 mL) was hydrogenated (50 Psi) at 25° C. with wet Pt/C (240 mg, 10%, 0.1 eq.) as a catalyst for 12 hours. After uptake of H₂ (3 eq.), the catalyst was filtered off and the filtrate was evaporated. The intermediate 7-d was obtained (1.8 g, Yield 86%).

### Step 4: synthesis of 1-cyclopropyl-7-(trifluoromethyl)-1H-benzo[d]imidazol-2(3H)-one 7-e

To the intermediate 7-d (1.6 g, 7.4 mmol, 1 eq.) dissolved in dichloromethane (60 mL) at 0° C. triethylamine (4.49 g, 44.4 mmol, 6 eq.) was added. At 0° C. a solution of triphosgene (2.19 g, 7.4 mmol, 1 eq.) in dichloromethane (20 mL) under vigorous stirring was added dropwise. The ice bath was removed and the mixture was stirred at 25° C. overnight. The solution was washed with water, aqueous NaHCO₃, aqueous citric acid and brine, dried over Na₂SO₄. The solvent was removed under vacuum. The residue was dissolved in CH₃CN and some solid was precipitated. The solid was filtered, washed with CH₃CN. 140 mg of the intermediate 7-e was obtained (Yield 8%).

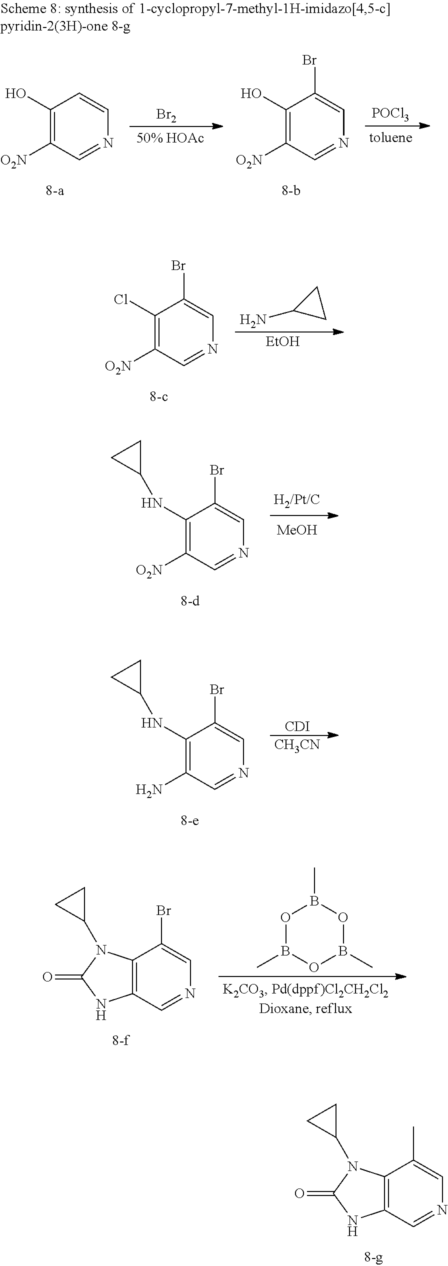

### Step 1: synthesis of 3-bromo-5-nitropyridin-4-ol 8-b

To a solution of 3-nitropyridin-4-ol (20 g, 142.76 mmol, 1 eq.) in 50% aqueous acetic acid (250 mL) bromine (113 g, 713 mmol, 5 eq.) was added dropwise. The resulting mixture was stirred for 20 hours at room temperature. The resulting precipitate was filtered and washed with water. 25 g of intermediate 8-b was obtained.

### Step 2: synthesis of 3-bromo-4-chloro-5-nitropyridine 8-c

To a suspension of 8-b (25 g, 114.16 mol) in toluene (50 mL) was added POCl₃ (50 mL) at room temperature. The mixture was slowly heated to 100° C. and stirred overnight at that temperature. The mixture was cooled to room temperature and concentrated. To the resulting residue ice-water was carefully added then the resulting mixture was extracted with ethyl acetate. The organic layers were separated, washed with water and brine, dried over anhydrous Na₂SO₄ and evaporated. 25 g of the intermediate 8-c was obtained.

### Step 3: synthesis of 3-bromo-N-cyclopropyl-5-nitropyridin-4-amine 8-d

To a solution of intermediate 8-c (25 g 105.29 mmol, 1 eq.) in ethanol (250 mL) was added cyclopropylamine (9.02 g, 157.93 mmol, 1.5 eq.). The solution was warmed to 80° C. for 4 hours. The solvent was evaporated and water was added. The resulting mixture was extracted with dichloromethane (3×50 mL). The organic layer was washed with brine, dried with MgSO₄ and concentrated. 26 g of intermediate 8-d was obtained.

### Step 4: synthesis of 5-bromo-N¹-cyclopropylpyridine-3,4-diamine 8-e

A solution of intermediate 8-d (17 g, 65.87 mmol) in CH₃OH (200 mL) was hydrogenated (30 Psi) at 25° C. with wet Pt/C (1.7 g) as a catalyst for 15 hours. After uptake of H₂ (3 eq), the catalyst was filtered off. The combined filtrates were evaporated till dryness. 12 g of intermediate 8-e was obtained.

### Step 5: synthesis of 7-bromo-1-cyclopropyl-1H-imidazo[4,5-c]pyridin-2(3H)-one 8-f

Carbonyldiimidazole (8.96 g, 55.24 mmol) was added to a solution of intermediate 8-e (12 g, 52.61 mmol) in CH₃CN (200 mL) at 0° C. The reaction mixture was allowed to warm to 25° C. and stirred for 1 hour. The solid was collected by filtration and was washed with CH₃CN (15 mL) to afford intermediate 8-f as a white powder (8.5 g).

### Step 6: synthesis of 1-cyclopropyl-7-methyl-1H-imidazo[4,5-c]pyridin-2(3H)-one 8-g

The mixture of intermediate 8-f (7.5 g, 29.52 mmol), Trimethylboroxine (7.41 g, 59.04 mmol), K₂CO₃(12.24 g, 88.55 mmol) and [1,1-bis(diphenylphosphino)ferrocene]-palladium chloride (2.41 g, 2.95 mmol) in 1,4-dioxane (200 mL) was stirred at 115° C. overnight at N₂ atmosphere. The residue was purified by high performance liquid chromatography. The desired fraction was collected, evaporated to remove CH₃CN in vacuum and neutralized with saturated NaHCO₃ solution. The aqueous solution was extracted with CH₂Cl₂. The organic layer was dried, filtered and the solvent was evaporated. 501 mg intermediate 8-g was obtained.

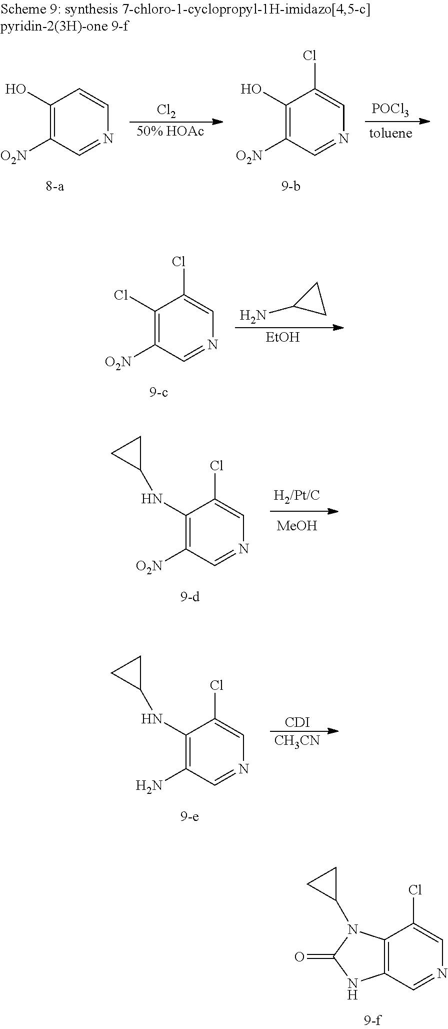

### Step 1: synthesis of 3-chloro-5-nitropyridin-4-ol 9-b

To a solution of 3-nitropyridin-4-ol 8-a (20 g, 142.76 mmol, 1 eq.) in 50% aqueous acetic acid (250 mL) chlorine was bubbled for 20 h at room temperature. The resulting precipitate was filtered and washed with water. The intermediate 9-b was obtained (24 g, 97%).

### Step 2: synthesis of 3,4-dichloro-5-nitropyridine 9-c

To a suspension of 9-b (35 g, 147.52 mol) in toluene (50 mL) was added POCl₃ (50 mL) at room temperature. The mixture was slowly heated to 100° C. and stirred overnight at that temperature. The mixture was cooled to room temperature and concentrated. To the resulting residue ice-water was carefully added then the resulting mixture was extracted with ethyl acetate. The organic layers were separated, washed with water and brine, dried over anhydrous Na₂SO₄ and evaporated. The intermediate 9-c was obtained (25 g, 90%).

### Step 3: synthesis of 3-chloro-N-cyclopropyl-5-nitropyridin-4-amine 9-d

To a solution of intermediate 9-c (25 g 125.94 mmol) in ethanol (250 mL) was added cyclopropylamine (11.10 g, 194.31 mmol). The solution was warmed to 80° C. for 1 hour. The solvent was evaporated and water was added. The resulting mixture was extracted with dichloromethane (3×50 mL). The organic layer was washed with brine, dried with MgSO₄ and concentrated. The intermediate 9-d was obtained (26 g, 94%).

### Step 4: synthesis of 5-chloro-N⁴-cyclopropylpyridine-3,4-diamine 9-e

A solution of intermediate 9-d (25 g, 117.03 mmol) in CH₃OH (200 mL) was hydrogenated (40 Psi) at 25° C. with wet Pt/C (1.7 g) as a catalyst for 15 hours. After uptake of H₂ (3 eq), the catalyst was filtered off. The combined filtrates were evaporated till dryness. The intermediate 9-e was obtained (21 g, 88%).

### Step 5: synthesis of 7-chloro-1-cyclopropyl-1H-imidazo[4,5-c]pyridin-2(3H)-one 9-f

Carbonyldiimidazole (19.47 g, 120.07 mmol) was added to a solution of intermediate 9-e (21 g, 114.36 mmol) in CH₃CN (200 mL) at 0° C. The reaction mixture was allowed to warm to 25° C. and stirred for 1 hour. The residue was purified by column chromatography over silica gel (eluent: CH₂Cl₂/CH₃OH 20/1) to afford the title intermediate 9-f as a white powder (11 g, 45%).

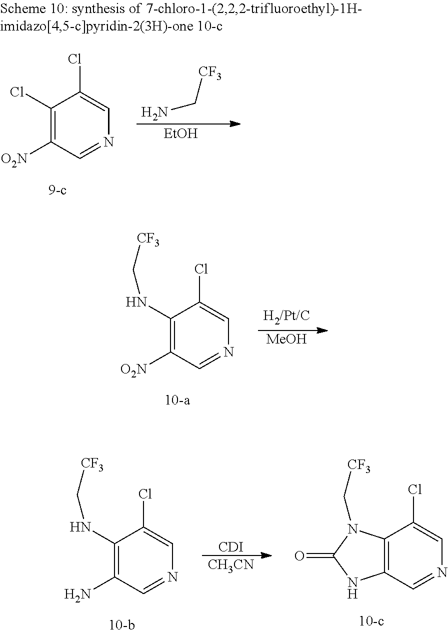

Intermediate 10-c was prepared by an analogous reaction protocol as intermediate 9-f using 2,2,2-trifluoroethyl amine and 3,4-dichloro-5-nitropyridine 9-c as starting material.

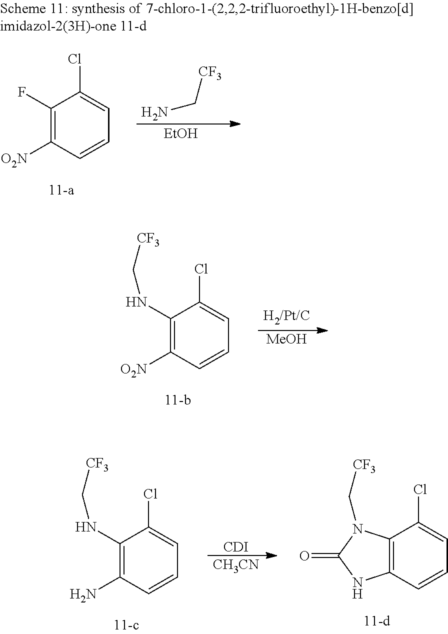

Intermediate 11-d was by an analogous reaction protocol as intermediate 6-d using 2,2,2-trifluoroethyl amine and 1-chloro-2-fluoro-3-nitrobenzene 11-a as starting material.

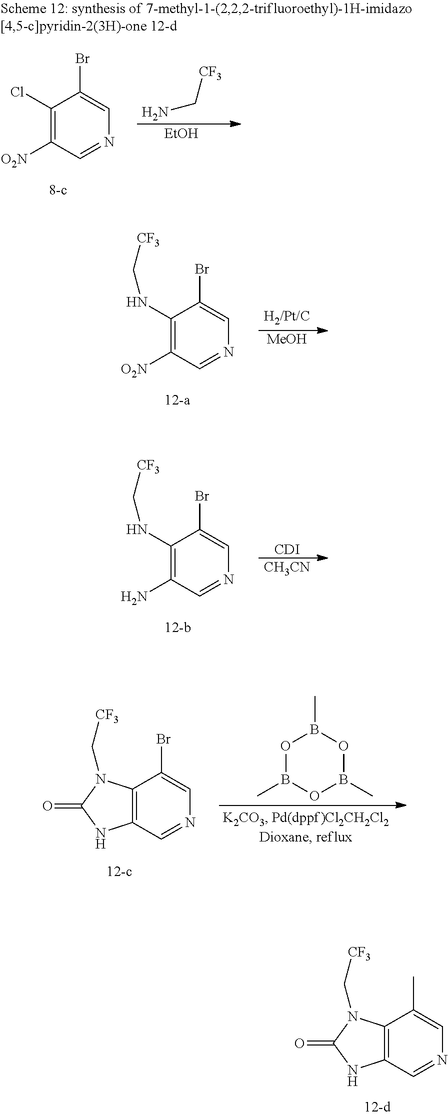

Intermediate 12-d was prepared by an analogous reaction protocol as intermediate 8-g using 2,2,2-trifluoroethyl amine and 3-bromo-4-chloro-5-nitropyridine 8-c as starting material.

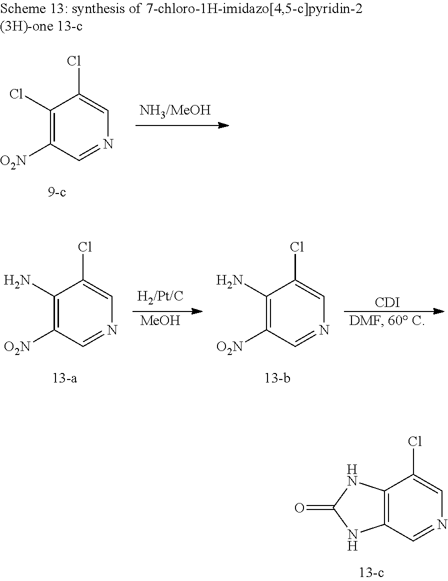

Intermediate 13-c was prepared by an analogous reaction protocol as intermediate 9-f using ammonia and 3-3,4-dichloro-5-nitropyridine 9-c as starting material.

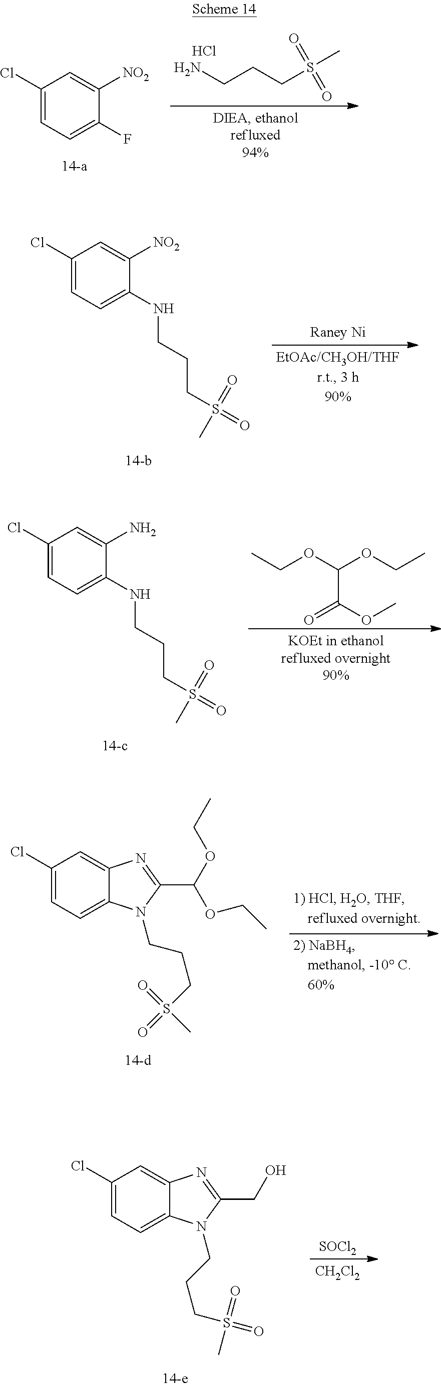

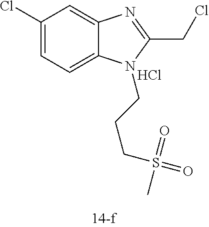 [SMILES: CS(=O)(=O)CCCn1c(CCl)nc2cc(Cl)ccc21 ; Cl]

Intermediate 14-a (7.6 g, 35 mmol), 3-(methylsulfonyl)propan-1-amine hydrochloride (6 g, 35 mmol) and diisopropylethylamine (DIEA) (13.5 g, 105 mmol) were dissolved in ethanol (70 mL) and refluxed for 14 h. The mixture was cooled to 20° C. The precipitate was filtered and washed with ethanol. 11 g (94%) of intermediate 14-b was obtained as an orange powder. Intermediate 14-b (10 g, 29.7 mmol) in methanol (200 mL), EtOAc (200 mL) and THF (200 mL) was hydrogenated with Raney Ni (10 g) as a catalyst at 20° C. (1 atm) for 3 h. After uptake of H₂ (3 eq), the catalyst was filtered off and the filtrate was evaporated. 10 g (90%) of intermediate 14-c was obtained as a black solid. Intermediate 14-c (10 g, 29.7 mmol) and methyl dimethoxy-acetate (9.2 g, 68.31 mmol) in 24 wt % KOEt in ethanol (13.5 g, 38.5 mmol) were stirred and refluxed overnight. The mixture was evaporated under vacuum. Water (200 mL) was added. Acetic acid was added to neutralize the mixture. The mixture was extracted with ethyl acetate (2×100 mL). The combined organic layers were washed with saturated NaHCO₃, brine and dried over Na₂SO₄. The solvent was removed under vacuum to yield 12.3 g (90%) of intermediate 14-d as dark oil. Intermediate 14-d (12.3 g, 29.3 mmol) in THF (100 mL) was stirred for 0.5 h at 20° C. to dissolve. Conc. HCl (21 mL) and H₂O (42 mL) were added. The mixture was refluxed for 6 h and then cooled to −10° C. CH₃OH (50 mL) were added, followed by careful addition of NaBH₄ (24 g, 629 mmol). The mixture was stirred for 0.5 h at 10° C. and concentrated under vacuum. Water (200 mL) was added. The mixture was extracted with ethyl acetate (2×100 mL). The combined organic layers were washed with brine and dried over Na₂SO₄. The solvent was removed under vacuum. The resulting solid was washed with ethyl acetate (2×5 mL) and dried under vacuum. 6.8 g (60%) of intermediate 14-e was obtained as an off-white solid.

¹H NMR (400 MHz, DMSO-d₆) δ ppm 2.20 (dq, J=7.8, 7.5 Hz, 2H), 2.98 (s, 3H), 3.16-3.24 (m, 2H), 4.42 (t, J=7.4 Hz, 2H), 4.73 (d, J=6.0 Hz, 2H), 5.73 (t, J=5.8 Hz, 1H), 7.42 (dd, J=8.7, 1.9 Hz, 1H), 7.63 (d, J=8.5 Hz, 1H), 7.79-7.83 (m, 1H)

To a solution of alcohol 14-e (363 mg, 1.414 mmole) in 30 mL of dichloromethane was added dropwise a solution of thionyl chloride (336 mg, 2 eq) in 10 mL of dichloromethane. The reaction mixture was stirred for one hour at 45° C. It was then concentrated under vacuum to give the desired intermediate 14-f (440 mg, 99%) as an HCl salt, which was used as such in the next step.

### Synthesis of Compounds

### Example 1

A detailed description of the synthesis of 7-Chloro-3-((5-chloro-1-(3-(methylsulfonyl)-propyl)-1H-benzo[d]imidazol-2-yl)methyl)-1-cyclopropyl-1H-imidazo[4,5-c]pyridin-2(3H)-one (P1), a representative example of the invention is given in Scheme 15.

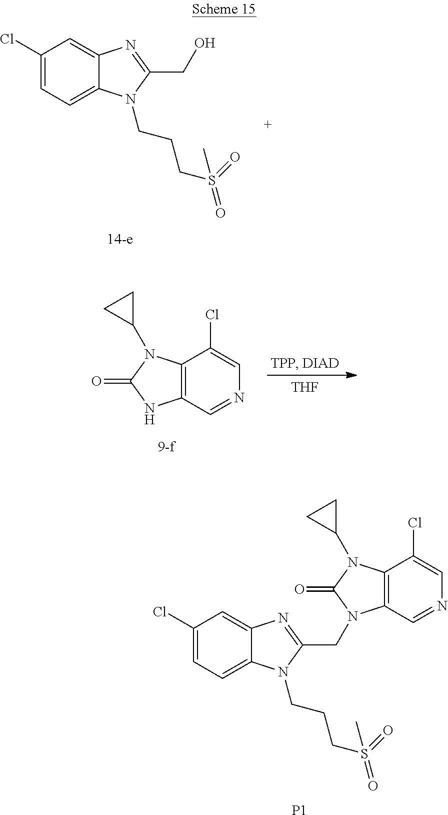 [SMILES: CS(=O)(=O)CCCn1c(CO)nc2cc(Cl)ccc21 ; O=c1[nH]c2cncc(Cl)c2n1C1CC1 ; CS(=O)(=O)CCCn1c(Cn2c(=O)n(C3CC3)c3c(Cl)cncc32)nc2cc(Cl)ccc21 ; "THF" ; "P1"]

In a 100 mL dry flask, 14-e (500 mg, 1.5 mmol), triphenylphosphine (TPP) (483 mg, 1.8 mmol, 1.2 eq) and intermediate 9-f (386 mg, 1.8 mmol, 1.2 eq) were dissolved in tetrahydrofuran (THF) (60 mL). The solution was placed under N₂ atmosphere and diisopropylazodicarboxylate (DIAD) (0.448 mL, 2.3 mmol, 1.5 eq) was added via syringe. The reaction mixture was stirred at room temperature under nitrogen overnight. The mixture was evaporated to dryness and purified by preparative HPLC on an RP Vydac Denali C18 column (10 μm, 250 g, 5 cm) using a 0.25% NH₄HCO₃ in H₂O/CH₃CN solution as the eluent. After evaporation and drying in vacuo, 570 mg (75%) of a white solid was obtained.

m/z=494 (M+H)⁺ (LCMS method 1)

¹H NMR (400 MHz, DMSO-d₆) δ ppm 1.11 (d, J=5.3 Hz, 4H), 2.16 (t, J=7.5 Hz, 2H), 3.00 (s, 3H), 3.13-3.19 (m, 1H), 3.20-3.27 (m, 2H), 4.48 (t, J=7.4 Hz, 2H), 5.43 (s, 2H), 7.31 (dd, J=8.7, 1.9 Hz, 1H), 7.64-7.74 (m, 2H), 8.24 (s, 1H), 8.41 (s, 1H)

### Example 2

A detailed description of the synthesis of 4-Chloro-1-((5-chloro-1-(3-(methylsulfonyl)propyl)-1H-benzo[d]imidazol-2-yl)methyl)-3-cyclopropyl-1H-benzo[d]imidazol-2(3H)-one (P2), a representative example of the invention is given in Scheme 16.

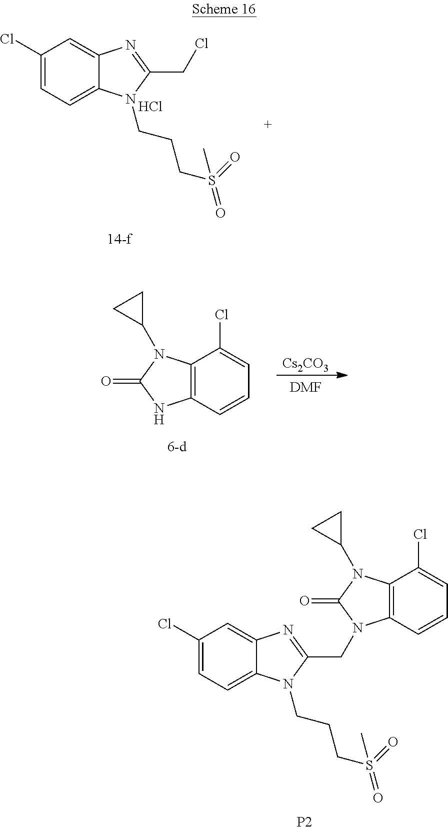

In a 100 mL dry flask, intermediate 14-f (500 mg, 1.4 mmol), intermediate 6-d (350 mg, 1.7 mmol) were dissolved in DMF (50 mL). The resulting mixture was stirred at room temperature then cesium carbonate (1 g, 3 mmol) was added. The reaction mixture was stirred at room temperature overnight. The resulting mixture was poured in iced-water then dichloromethane was added and the water layer was extracted with dichloromethane. The organic layer was dried over MgSO4 and concentrated. The residue was triturated with dichloromethane and ether and the resulting white solid was dried in the oven (560 mg, 81%).

m/z=493 (M+H)⁺ (LCMS Method 1)

¹H NMR (400 MHz, DMSO-d₆) δ ppm 1.05-1.18 (m, 4H), 2.11 (m, J=7.5, 7.5 Hz, 2H), 3.00 (s, 3H), 3.10-3.18 (m, 1H), 3.18-3.26 (m, 2H), 4.48 (t, J=7.5 Hz, 2H), 5.36 (s, 2H), 7.01-7.07 (m, 1H), 7.07-7.11 (m, 1H), 7.21 (dd, J=7.7, 1.1 Hz, 1H), 7.31 (dd, J=8.8, 2.0 Hz, 1H), 7.65 (d, J=2.0 Hz, 1H), 7.67 (d, J=8.5 Hz, 1H)

### Example 3

### Synthesis of 1-((5-Chloro-1-(3-(methylsulfonyl)propyl)-1H-benzo[d]imidazol-2-yl)-methyl)-3-cyclopropyl-4-methyl-1H-benzo[d]imidazol-2(3H)-one (P3)

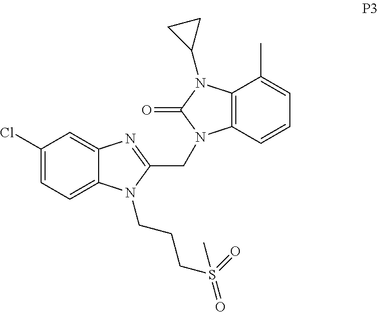 [SMILES: Cc1cccc2c1n(C1CC1)c(=O)n2Cc1nc2cc(Cl)ccc2n1CCCS(C)(=O)=O]

Compound P3 was prepared by an analogous reaction protocol as compound P2 using intermediate 14-f and 1-cyclopropyl-7-methyl-1H-benzo[d]imidazol-2(3H)-one 5-d as starting material.

m/z=473 (M+H)⁺ (LCMS Method 2)

¹H NMR (400 MHz, DMSO-d₆) δ ppm 1.08 (d, J=5.3 Hz, 4H), 1.98-2.12 (m, 2H), 2.68 (s, 3H), 2.98 (s, 3H), 3.13-3.23 (m, 3H), 4.46 (t, J=7.5 Hz, 2H), 5.31 (s, 2H), 6.80-6.87 (m, 1H), 6.91 (t, J=7.7 Hz, 1H), 7.04 (d, J=7.5 Hz, 1H), 7.30 (dd, J=8.8, 2.0 Hz, 1H), 7.66 (dd, J=5.3, 3.3 Hz, 2H)

### Example 4

### Synthesis of 3-((5-chloro-1-(3-(methylsulfonyl)propyl)-1H-benzo[d]imidazol-2-yl)methyl)-1-cyclopropyl-7-methyl-1H-imidazo[4,5-c]pyridin-2(3H)-one (P4)

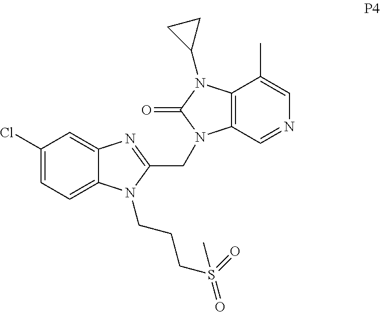 [SMILES: Cc1cncc2c1n(C1CC1)c(=O)n2Cc1nc2cc(Cl)ccc2n1CCCS(C)(=O)=O]

Compound P4 was prepared by an analogous reaction protocol as compound P1 using intermediate 14-e and 1-cyclopropyl-7-methyl-1H-imidazo[4,5-c]pyridin-2(3H)-one 8-g as starting material.

m/z=474 (M+H)⁺ (LCMS Method 1)

1H NMR (400 MHz, DMSO-d6) δ ppm 1.09 (d, J=5.28 Hz, 4H) 2.04-2.17 (m, 2H) 2.65 (s, 3H) 3.00 (s, 3H) 3.16-3.25 (m, 3H) 4.47 (t, J=7.37 Hz, 2H) 5.39 (s, 2H) 7.31 (dd, J=8.58, 1.98 Hz, 1H) 7.63-7.73 (m, 2H) 8.03 (s, 1H) 8.27 (s, 1H)

### Example 5

### Synthesis of 1-((5-chloro-1-(3-(methylsulfonyl)propyl)-1H-benzo[d]imidazol-2-yl)-methyl)-3-cyclopropyl-4-(trifluoromethyl)-1H-benzo[d]imidazol-2(3H)-one (P5)

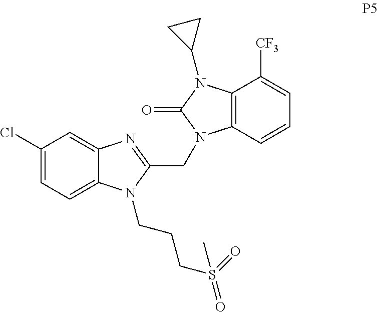 [SMILES: CS(=O)(=O)CCCn1c(Cn2c(=O)n(C3CC3)c3c(C(F)(F)F)cccc32)nc2cc(Cl)ccc21]

Compound P5 was prepared by an analogous reaction protocol as compound P2 using intermediate 14-f and 1-cyclopropyl-7-(trifluoromethyl)-1H-benzo[d]imidazol-2(3H)-one 7-e as starting material.

m/z=527 (M+H)⁺ (LCMS Method 1)

MP=210.18° C.

1H NMR (400 MHz, DMSO-d6) δ ppm 0.93-1.02 (m, 2H) 1.02-1.08 (m, 2H) 2.08-2.23 (m, 2H) 3.00 (s, 3H) 3.04-3.12 (m, 1H) 3.19-3.27 (m, 2H) 4.50 (t, J=7.37 Hz, 2H) 5.43 (s, 2H) 7.25 (t, J=8.10 Hz, 1H) 7.31 (dd, J=8.58, 1.76 Hz, 1H) 7.43 (d, J=7.92 Hz, 1H) 7.57 (d, J=7.92 Hz, 1H) 7.64 (d, J=1.54 Hz, 1H) 7.68 (d, J=8.58 Hz, 1H)

### Example 6

### Synthesis of 7-chloro-3-((5-chloro-1-(3-(methylsulfonyl)propyl)-1H-benzo[d]imidazol-2-yl)methyl)-1-(2,2,2-trifluoroethyl)-1H-imidazo[4,5-c]pyridin-2(3H)-one (P6)

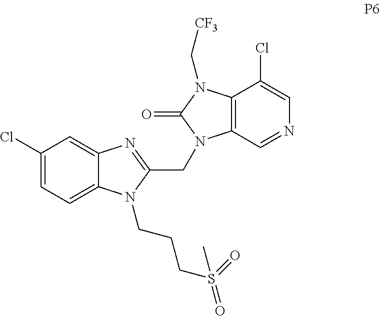 [SMILES: CS(=O)(=O)CCCn1c(Cn2c(=O)n(CC(F)(F)F)c3c(Cl)cncc32)nc2cc(Cl)ccc21]

Compound P6 was prepared in the same manner as compound P1 using intermediate 14-e and 7-chloro-1-(2,2,2-trifluoroethyl)-1H-imidazo[4,5-c]pyridin-2(3H)-one 10-c as starting material.

m/z=536 (M+H)⁺ (LCMS Method 1)

MP=232.82° C.

1H NMR (400 MHz, DMSO-d6) δ ppm 2.13-2.25 (m, 2H) 3.01 (s, 3H) 3.21-3.28 (m, 2H) 4.50 (t, J=7.48 Hz, 2H) 5.03 (q, J=8.80 Hz, 2H) 5.57 (s, 2H) 7.32 (dd, J=8.58, 1.98 Hz, 1H) 7.64 (d, J=1.76 Hz, 1H) 7.70 (d, J=8.58 Hz, 1H) 8.33 (s, 1H) 8.51 (s, 1H)

### Example 7

### Synthesis of 4-chloro-1-((5-chloro-1-(3-(methylsulfonyl)propyl)-1H-benzo[d]imidazol-2-yl)methyl)-3-(2,2,2-trifluoroethyl)-1H-benzo[d]imidazol-2(3H)-one (P7)

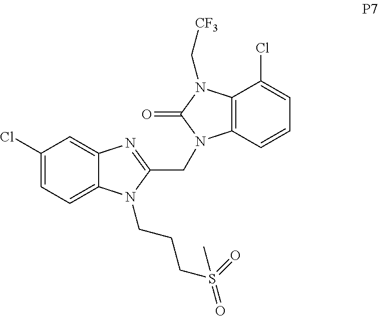 [SMILES: CS(=O)(=O)CCCn1c(Cn2c(=O)n(CC(F)(F)F)c3c(Cl)cccc32)nc2cc(Cl)ccc21]

Compound P7 was prepared by an analogous reaction protocol as compound P2 using intermediate 14-f and 7-chloro-1-(2,2,2-trifluoroethyl)-1H-benzo[d]imidazol-2(3H)-one 11-d as starting material.

m/z=535 (M+H)⁺ (LCMS Method 3)

1H NMR (400 MHz, DMSO-d6) δ ppm 2.09-2.20 (m, 2H) 3.01 (s, 3H) 3.20-3.27 (m, 2H) 4.49 (t, J=7.48 Hz, 2H) 5.04 (q, J=8.80 Hz, 2H) 5.50 (s, 2H) 7.09-7.21 (m, 2H) 7.28-7.34 (m, 2H) 7.64 (d, J=1.98 Hz, 1H) 7.69 (d, J=8.80 Hz, 1H)

Antiviral Activity Black 96-well clear-bottom microtiter plates (Corning, Amsterdam, The Netherlands) were filled in duplicate using a customized robot system with serial 4-fold dilutions of compound in a final volume of 50 μl culture medium [RPMI medium without phenol red, 10% FBS, 0.04% gentamycin (50 mg/ml) and 0.5% DMSO]. Then, 100 μl of a HeLa cell suspension (5×10⁴ cells/ml) in culture medium was added to each well followed by the addition of 50 μl rgRSV224 (MOI=0.02) virus in culture medium using a multidrop dispenser (Thermo Scientific, Erembodegem, Belgium). rgRSV224 virus is an engineered virus that includes an additional GFP gene (Hallak et al, 2000) and was in-licensed from the NIH (Bethesda, Md., USA). Medium, virus- and mock-infected controls were included in each test. Cells were incubated at 37° C. in a 5% CO₂ atmosphere. Three days post-virus exposure, viral replication was quantified by measuring GFP expression in the cells by a MSM laser microscope (Tibotec, Beerse, Belgium). The EC₅₀ was defined as the 50% inhibitory concentration for GFP expression. In parallel, compounds were incubated for three days in a set of white 96-well microtiter plates (Corning) and the cytotoxicity of compounds in HeLa cells was determined by measuring the ATP content of the cells using the ATPlite kit (PerkinElmer, Zaventem, Belgium) according to the manufacturer's instructions. The CC₅₀ was defined as the 50% concentration for cytotoxicity.

### REFERENCES

Hallak L K, Spillmann D, Collins P L, Peeples M E. Glycosaminoglycan sulfation requirements for respiratory syncytial virus infection. J. Virol. 740, 10508-10513 (2000).

Compounds were tested for RSV inbitory activity. The results are depicted in the Table below (n.d. means not determined):

| | | WT activity | Tox |
|---|---|---|---|
| | Structure | EC₅₀ (μM) | CC₅₀ (μM) |
| P1 | 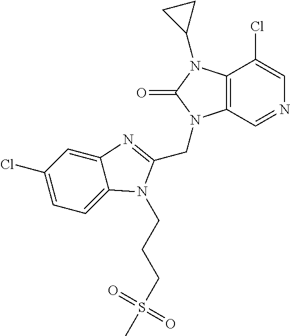 [SMILES: CS(=O)(=O)CCCn1c(Cn2c(=O)n(C3CC3)c3c(Cl)cncc32)nc2cc(Cl)ccc21] | 0.0017 | >100 |
| P2 | 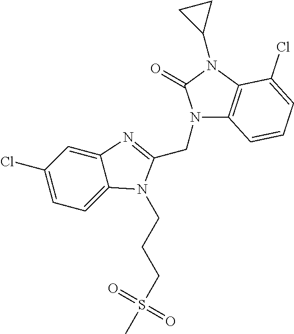 [SMILES: CS(=O)(=O)CCCn1c(Cn2c(=O)n(C3CC3)c3c(Cl)cccc32)nc2cc(Cl)ccc21] | 0.0074 | >100 |
| P3 | 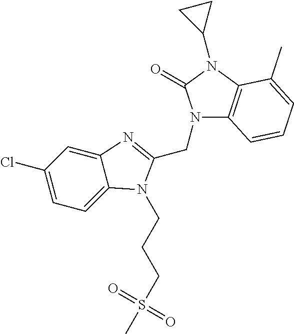 [SMILES: Cc1cccc2c1n(C1CC1)c(=O)n2Cc1nc2cc(Cl)ccc2n1CCCS(C)(=O)=O] | 0.0051 | 64.63 |
| P4 | 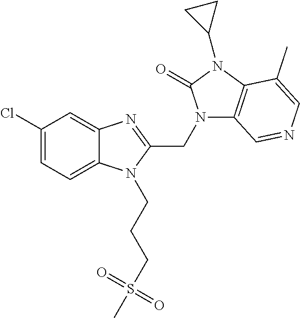 [SMILES: Cc1cncc2c1n(C1CC1)c(=O)n2Cc1nc2cc(Cl)ccc2n1CCCS(C)(=O)=O] | 0.0014 | >100 |
| P5 | 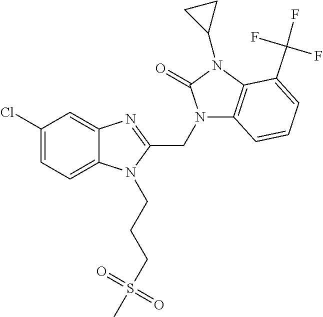 [SMILES: CS(=O)(=O)CCCn1c(Cn2c(=O)n(C3CC3)c3c(C(F)(F)F)cccc32)nc2cc(Cl)ccc21] | 0.028 | >100 |
| P6 | 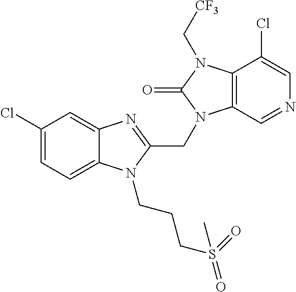 [SMILES: CS(=O)(=O)CCCn1c(Cn2c(=O)n(CC(F)(F)F)c3c(Cl)cncc32)nc2cc(Cl)ccc21] | 0.009 | >100 |
| P7 | 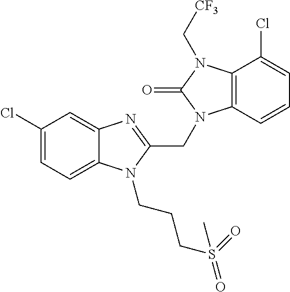 [SMILES: CS(=O)(=O)CCCn1c(Cn2c(=O)n(CC(F)(F)F)c3c(Cl)cccc32)nc2cc(Cl)ccc21] | 0.012 | 69 |

### Composition Examples

“Active ingredient” (a.i.) as used throughout these examples relates to a compound of Formula (I), including any tautomer or stereoisomeric form thereof, or a pharmaceutically acceptable addition salt or a solvate thereof; in particular to any one of the exemplified compounds.

Typical examples of recipes for the formulation of the invention are as follows:

### 1. Tablets

| | |
|---|---|
| Active ingredient | 5 to 50 mg |
| Di-calcium phosphate | 20 mg |
| Lactose | 30 mg |
| Talcum | 10 mg |
| Magnesium stearate | 5 mg |
| Potato starch | ad 200 mg |

### 2. Suspension

An aqueous suspension is prepared for oral administration so that each milliliter contains 1 to 5 mg of active ingredient, 50 mg of sodium carboxymethyl cellulose, 1 mg of sodium benzoate, 500 mg of sorbitol and water ad 1 ml.

### 3. Injectable

A parenteral composition is prepared by stirring 1.5% (weight/volume) of active ingredient in 0.9% NaCl solution or in 10% by volume propylene glycol in water.

### 4. Ointment

| | |
|---|---|
| Active ingredient | 5 to 1000 mg |
| Stearyl alcohol | 3 g |
| Lanoline | 5 g |
| White petroleum | 15 g |
| Water | ad 100 g |

In this Example, active ingredient can be replaced with the same amount of any of the compounds according to the present invention, in particular by the same amount of any of the exemplified compounds.

Reasonable variations are not to be regarded as a departure from the scope of the invention. It will be obvious that the thus described invention may be varied in many ways by those skilled in the art.

1. A compound of Formula (I), 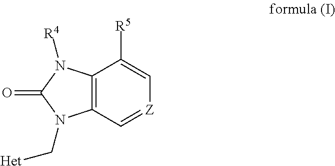
a tautomer or a stereoisomeric form thereof, wherein
Het is a heterocycle having formula (a) 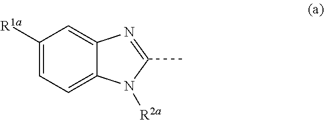
R^{1a} is Br or Cl;
R^{2a} is —(CR^{8a}R^{9a})ₙ—R^{10a};
each R^{8a} and R^{9a} are independently chosen from the group consisting of H, C₁-C₁₀alkyl and C₃-C₇cycloalkyl; or R^{8a} and R^{9a} taken together form a 4 to 6 membered aliphatic ring; wherein the 4 to 6 membered aliphatic ring optionally contains one or more heteroatoms selected from the group consisting of N, S and O;
R^{10a} is selected from the group consisting of H, C₁-C₆alkyl, R¹¹, OH, CF₃, CHF₂, F, Cl, SO₂CH₃, SO₂C₃-C₇cycloalkyl, NR^{8a}SO₂R^{8a}, SO₂NR^{8a}R^{9a}, NR^{8a}SO₂C₃-C₇cycloalkyl, CN, NR^{8a}R^{9a}, COOH, COOR^{8a}, CONR^{8a}R^{9a}, OCOC₁-C₆alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from the group consisting of N, S and O;
R¹¹ is selected from the group consisting of C₁-C₆ alkyl, C₃-C₇cycloalkyl, phenyl, pyridinyl and pyrazolyl; each substituted with one or more substituents each independently selected from the group consisting of CF₃, CH₃, OCH₃, OCF₃ and halogen;
n is an integer having a value from 1 to 6;
R⁵ is selected from the group consisting of C₁-C₆alkyl, C₁-C₆alkyloxy, CN, CF₃ and halo;
R⁴ is selected from the group consisting of hydrogen, tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het¹, Het² and C₃-C₇cycloalkyl substituted with one or more substituents selected from the group consisting of halo and C₁-C₄alkyl;
aryl represents phenyl or naphthalenyl; said aryl optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a}, and C₁-C₄alkyl;
Het¹ represents a monocyclic 4 to 6 membered non-aromatic heterocycle containing one or two heteroatoms each independently selected from the group consisting of O, S and N; or a bicyclic 7 to 11 membered non-aromatic heterocycle containing one or two heteroatoms each independently selected from the group consisting of O, S and N; said Het¹ optionally being substituted with one or more substituents each independently selected from the group consisting of halo, C₁-C₄alkyloxy, SO₂R^{8a}, C₁-C₄alkylcarbonyl, C₁-C₄alkyloxycarbonyl, CO(aryl), COHet², pyridinyl, CF₃, SO₂N(C₁-C₄alkyl)₂, SO₂NH(C₁-C₄alkyl), NH(C═O)(C₁₋₄alkyl), (C═O)NH(C₁₋₄alkyl), (C═S)NH(C₁₋₄alkyl), C₁-C₄alkyl and C₁-C₄alkyl substituted with one hydroxy;
Het² represents a monocyclic 5 to 6 membered aromatic heterocycle containing one or more heteroatoms each independently selected from the group consisting of O, S and N; or a bicyclic 8 to 12 membered aromatic heterocycle containing one or more heteroatoms each independently selected from the group consisting of O, S and N; said Het² optionally being substituted with one or more substituents each independently selected from the group consisting of halo, C₁-C₄alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, OCOR^{8a}, NR^{8a}SO₂R^{9a}, SO₂NR^{8a}R^{9a}, SO₂R^{8a}, OCONR^{8a}R^{9a}, OCONR^{8a}R^{11a}, N(R^{8a})CON(R^{8a}R^{9a}), N(R^{8a})COOR^{11a} and C₁-C₄alkyl;
R^{11a} is selected from the group consisting of phenyl, pyridinyl and pyrazolyl; each optionally substituted with one or more substituents each independently selected from the group consisting of CF₃, CH₃, OCH₃, OCF₃ and halogen;
or R^{11a} is C₁-C₆ alkyl or C₃-C₇cycloalkyl; each substituted with one or more substituents each independently selected from the group consisting of CF₃, CH₃, OCH₃, OCF₃ and halogen;
Z is CH or N;
or a pharmaceutically acceptable addition salt or a solvate thereof.

2. The compound according to claim 1 wherein R⁴ is selected from the group consisting of tert-butyl, C₃-C₇cycloalkyl, CH(CH₃)(CF₃), C₂-C₁₀alkenyl, CH₂CF₃, SO₂CH₃, —CH₂-p-fluorophenyl, aryl, Het % Het² and C₃-C₇cycloalkyl substituted with one or more substituents selected from the group consisting of halo and C₁-C₄alkyl.

3. The compound according to claim 2, wherein R⁴ is selected from the group consisting of C₃-C₇cycloalkyl, CH₂CF₃ and C₃-C₇cycloalkyl substituted with one or more substituents selected from the group consisting of halo and C₁-C₄alkyl.

4. The compound according to claim 1 wherein each R^{8a} and R^{9a} are independently chosen from the group consisting of H, C₁-C₁₀alkyl and C₃-C₇cycloalkyl.

5. The compound according to claim 1, wherein
Het is a heterocycle having formula (a)
R^{1a} is Br or Cl;
R^{2a} is —(CR^{8a}R^{9a})ₙ—R^{10a};
each R^{8a} and R^{9a} are independently chosen from the group consisting of hydrogen and C₁-C₁₀alkyl;
R^{10a} is selected from the group consisting of SO₂CH₃, SO₂C₃-C₇cycloalkyl, SO₂NR^{8a}R^{9a};
n is an integer having a value from 1-6;
R⁵ is selected from the group consisting of C₁-C₆alkyl, CF₃ and halo;
R⁴ is selected from the group consisting of C₃-C₇cycloalkyl, CH₂CF₃ and C₃-C₇cycloalkyl substituted with one or more substituents selected from the group consisting of halo and C₁-C₄alkyl;
Z is CH or N.

6. The compound according to claim 1, wherein
Het is a heterocycle having formula (a)
R^{1a} is Cl;
R^{2a} is —(CR^{8a}R^{9a})ₙ—R^{10a};
R^{8a} and R^{9a} are hydrogen;
R^{10a} is SO₂CH₃;
n is 3;
R⁵ is selected from the group consisting of methyl, CF₃ and chloro;
R⁴ is selected from the group consisting of cyclopropyl and CH₂CF₃;
Z is CH or N.

7. The compound according to claim 1, wherein R^{1a} is chloro.

8. The compound according to claim 1, wherein Z is N.

9. The compound according to claim 1, wherein Z is CH.

10. The compound according to claim 1 wherein R⁵ is selected from the group consisting of C₁-C₆alkyl, halogen and CF₃.

11. The compound according to claim 1 wherein R⁴ is cyclopropyl.

12. The compound according to claim 1, wherein the compound is selected from the group consisting of 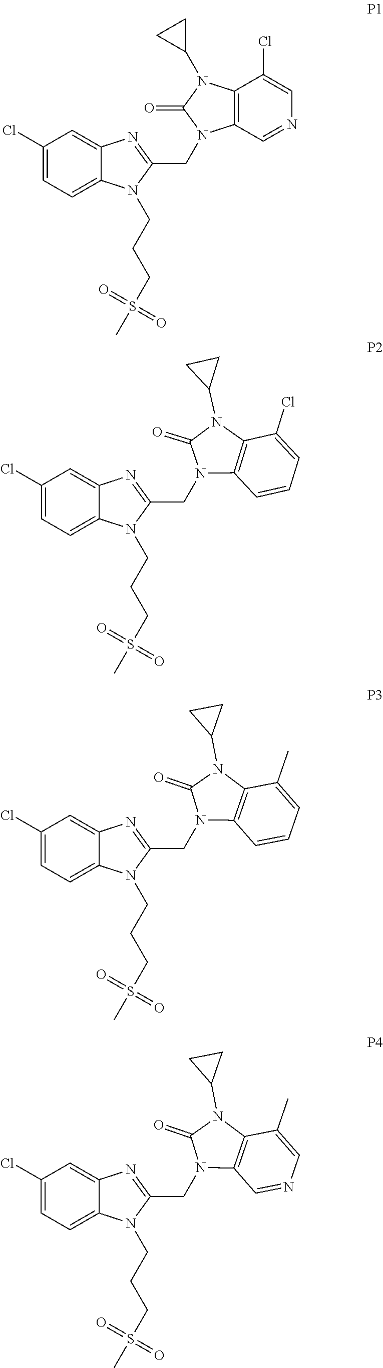 [SMILES: CS(=O)(=O)CCCn1c(Cn2c(=O)n(C3CC3)c3c(Cl)cncc32)nc2cc(Cl)ccc21 ; CS(=O)(=O)CCCn1c(Cn2c(=O)n(C3CC3)c3c(Cl)cccc32)nc2cc(Cl)ccc21 ; Cc1cccc2c1n(C1CC1)c(=O)n2Cc1nc2cc(Cl)ccc2n1CCCS(C)(=O)=O ; Cc1cncc2c1n(C1CC1)c(=O)n2Cc1nc2cc(Cl)ccc2n1CCCS(C)(=O)=O] 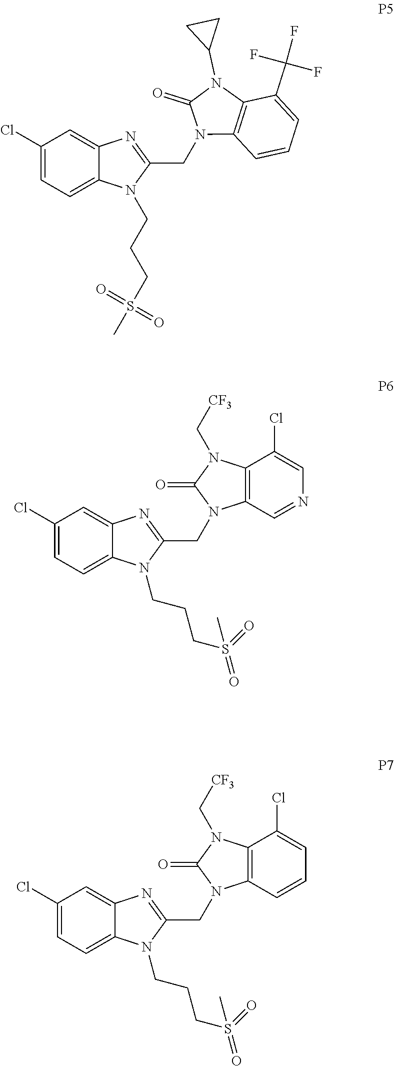 [SMILES: CS(=O)(=O)CCCn1c(Cn2c(=O)n(C3CC3)c3c(C(F)(F)F)cccc32)nc2cc(Cl)ccc21 ; CS(=O)(=O)CCCn1c(Cn2c(=O)n(CC(F)(F)F)c3c(Cl)cncc32)nc2cc(Cl)ccc21 ; CS(=O)(=O)CCCn1c(Cn2c(=O)n(CC(F)(F)F)c3c(Cl)cccc32)nc2cc(Cl)ccc21]
and tautomers and stereoisomeric forms thereof,
and pharmaceutically acceptable addition salts and solvates thereof.

13. (canceled)

14. A pharmaceutical composition comprising a pharmaceutically acceptable carrier, and as active ingredient a therapeutically effective amount of a compound as defined in claim 1.

15. (canceled)

16. A method of treating a respiratory syncytial viral (RSV) infection comprising administering to a subject in need of treatment an anti-virally effective amount of a compound as claimed in claim 1.
